(19) United States
(12) Patent Application Publication
(10) Pub. No.: US 2024/0122530 A1
(43) Pub. Date: Apr. 18, 2024

(54) WEARABLE ULTRASOUND AND PHOTOACOUSTIC DEVICE FOR FETAL AND/OR LABOR MONITORING

(71) Applicant: The Johns Hopkins University, Baltimore, MD (US)

(72) Inventors: Jeeun KANG, Baltimore, MD (US); Raymond C. KOEHLER, Baltimore, MD (US); Ernest M. GRAHAM, Clarksville, MD (US); Emad M. BOCTOR, Ellicott City, MD (US); Jennifer LEE-SUMMERS, Baltimore, MD (US)

(73) Assignee: The Johns Hopkins University, Baltimore, MD (US)

(21) Appl. No.: 18/543,277

(22) Filed: Dec. 18, 2023

Related U.S. Application Data
(63) Continuation-in-part of application No. 17/309,382, filed on May 24, 2021.
(60) Provisional application No. 62/771,410, filed on Nov. 26, 2018.

(51) Int. Cl. A61B 5/00

(52) U.S. Cl. CPC A61B 5/4362; A61B 5/0095; A61B 5/4064; A61B 5/6823; A61B 2503/02; A61B 2503/045; A61B 2560/0214

(57) ABSTRACT

The present disclosure is associated with monitoring health of a patient. An example photoacoustic monitoring device includes a light-guiding component to guide light energy toward tissue to cause the light energy to be absorbed by the tissue; an ultrasound transmission component to transmit acoustic energy toward the tissue to cause a biological response from the tissue; and a sensing component to perform one or more of ultrasound or photoacoustic imaging to sense the biological response from the tissue and permit a status of the tissue to be determined. In some implementations, the biological response is sensed based on the light energy absorbed by the tissue during the biological response caused by the acoustic energy transmitted toward the tissue.

### RELATED APPLICATIONS

This application is a continuation-in-part based on U.S. Nonprovisional patent application Ser. No. 17/309,382, filed on May 24, 2021, entitled “APPARATUS AND METHOD FOR PATIENT MONITORING BASED ON ULTRASOUND MODULATION,” which is a 371 national stage of PCT Application No. PCT/US2019/063084, filed on Nov. 25, 2019, entitled “APPARATUS AND METHOD FOR PATIENT MONITORING BASED ON ULTRASOUND MODULATION,” which claims priority to U.S. Provisional Patent Application No. 62/771,410, filed on Nov. 26, 2018, entitled “APPARATUS AND METHOD FOR PHOTOACOUSTIC MONITORING BASED ON ULTRASOUND NEUROMODULATION,” the contents of which are incorporated by reference herein in their entirety.

### GOVERNMENT LICENSE RIGHTS

This invention was made with U.S. Government support under grant 1R01HL139543-01, awarded by the National Heart, Lung, and Blood Institute, and grant 3U54EB015408-09D3, awarded by the National Institute of Biomedical Imaging and Bioengineering. The U.S. Government has certain rights in the invention.

### BACKGROUND

Hypoxic-ischemic encephalopathy (HIE) is a brain injury caused by oxygen deprivation to the brain. Perinatal HIE remains a significant cause of developmental brain injury despite advances in obstetric and neonatal medicine. For example, due to inadequate blood flow through the placenta, blood flow to a fetal brain can be impeded, preventing delivery of oxygen in the blood from reaching the brain. In some cases, blood flow to the uterus or through the umbilical cord may be impeded and result in a decrease in oxygenation to the entire body of the fetus. In other cases, a blood clot can be transported into one of the cerebral arteries of the fetus or newborn and cause a stroke. Accordingly, before labor, during labor and/or after labor, one or more health characteristics of the fetus or newborn may be monitored to attempt to predict and/or detect potential perinatal HIE. Other types of biological conditions, such as internal bleeding, and/or the like, may similarly be monitored or detected to preserve the health of the fetus, newborn, or other type of patient.

### SUMMARY

According to some implementations, a device for noninvasive biological function monitoring includes a light-guiding component to guide light energy toward tissue to cause the light energy to be absorbed by the tissue; an ultrasound transmission component to transmit acoustic energy toward the tissue to cause a biological response from the tissue; and a sensing component to perform one or more of ultrasound or photoacoustic imaging to sense the biological response from the tissue and permit a status of the tissue to be determined, wherein the biological response is sensed based on the light energy absorbed by the tissue during the biological response caused by the acoustic energy transmitted toward the tissue.

According to some implementations, a system for biological function monitoring includes a photoacoustic monitoring device, comprising a light source; a light-guiding component; an ultrasound transmission component; and a sensing component. In some implementations, the system includes a control device, wherein the control device includes one or more processors to control the light source to emit light energy as pulsed light energy at multiple near-infrared range (NIR) wavelengths, wherein the light-guiding component is arranged to guide the light energy emitted by the light source toward tissue of a patient; control the ultrasound transmission component to transmit acoustic energy toward the tissue to incite a biological response from the tissue; receive, from the ultrasound sensing component, one or more of ultrasound or photoacoustic imaging data associated with the biological response, wherein the imaging data is representative of the light energy being absorbed by the tissue during the biological response; and generate an output that indicates the biological response from the tissue of the patient.

According to some implementations, a method for monitoring a biological function may include causing, by a control device, a light source of a monitoring device to emit pulsed light energy at multiple NIR wavelengths, wherein the light source is coupled to a light-guiding component that guides the light energy toward tissue of a patient to cause the light energy to be absorbed by the tissue; causing, by the control device, an ultrasound transmission component of the monitoring device to transmit acoustic energy toward the tissue to cause a biological response from the tissue; obtaining, by the control device and from a sensing component of the monitoring device, one or more of ultrasound or photoacoustic imaging data associated with the biological response, wherein the ultrasound or photoacoustic imaging data is generated from the light energy being absorbed by the tissue; and generating, by the control device, an output that indicates the biological response indicated in the ultrasound or photoacoustic imaging data.

### BRIEF DESCRIPTION OF THE DRAWINGS

FIG. 1 is a diagram of an example implementation described herein.

FIG. 2 is a diagram of an example implementation described herein.

FIGS. 3A-3C are diagrams of example implementations of an electromagnetic-evoked acoustic device described herein.

FIG. 4 is a diagram regarding an example implementation described herein.

FIGS. 5A-5G are diagrams of example implementations of a wearable ultrasound and photoacoustic device for fetal and/or labor monitoring.

FIG. 6 is a diagram of an example environment in which systems and/or methods described herein may be implemented.

FIG. 7 is a diagram of example components of one or more devices of FIG. 5.

FIGS. 8-10 are flowcharts of example processes associated with one or more example implementations described herein.

### DETAILED DESCRIPTION

The following detailed description of example implementations refers to the accompanying drawings. The same reference numbers in different drawings may identify the same or similar elements.

A child may be monitored before and/or after birth to detect any potential injury to the child (e.g., as a result of the labor) and/or a status (e.g., a health status) of the child. In some cases, a human actor may perform a direct examination of the child and determine a score (e.g., an Apgar score) associated with the health of the child. However, such an examination cannot be performed until after the birth of the child. In some instances, the heart rate of the child is monitored before and/or after birth to detect the flow of oxygen in the child's body. A lack of a sufficient flow of oxygen may be an indicator of developing hypoxic-ischemic encephalopathy (HIE). However, a heart rate of the child may fluctuate, before and/or after birth, for reasons other than HIE, and thus can provide a high false-positive rate for detecting abnormalities and/or predicting childhood neurologic injury. In some instances, magnetic resonance imaging (MRI) of a child can be a diagnostic tool to detect HIE after the birth of the child, but the MRI is generally not performed until 7-10 days after birth. In some instances, biophotonic techniques (e.g., near-infrared (NIR) spectroscopy (NIRS), diffused optical tomography, and/or the like) can be used to noninvasively monitor a cerebral oxygenation level and changes in blood flow within a brain of a child in real-time. However, such biophotonic techniques can only provide spatial resolution at the centimeter scale, which may be ineffective in detecting HIE or stroke before and/or after birth.

Some implementations described herein provide an electromagnetic-evoked (EM-evoked) device that can monitor a child before and after birth via ultrasound modulation of tissue of the child, and corresponding electromagnetic (EM) imaging of a biological response (e.g., a neural response) incited by the ultrasound modulation (e.g., neuromodulation of a neural response). As described herein, the EM-evoked device may include a photoacoustic device, and/or a thermoacoustic device that enables noninvasive monitoring of biological responses (e.g., a brain hemodynamic response, bleeding, and/or the like) to incited acoustic energy, which enables photoacoustic imaging and/or thermoacoustic imaging, respectively, of a severity of injury (e.g., neuronal injury, intra-abdominal injury, and/or the like) of the patient and/or determining a multi-spectral estimation of tissue oxyhemoglobin saturation. As described herein, EM imaging can provide spectral information of biological tissue. For example, photoacoustic imaging can provide relatively rich spectral information in a near-infrared range, while thermoacoustic imaging can employ less-attenuative microwave excitation for relatively deep imaging depth. In some implementations, the EM-evoked device (e.g., by emitting continuous energy, pulsed energy, and/or the like) enables monitoring of tissue (e.g., brain tissue) and one or more parameters over time (across one or more neural responses) that are indicative of HIE. In some implementations, the EM-evoked device may detect and/or determine timing associated with a neural response based on measured electrical activity associated with the neural response. Additionally, or alternatively, the electrical activity associated with the neural response may be used (e.g., in combination with ultrasound sensing and/or imaging) to determine a status of the tissue based on the neural response. Accordingly, the EM-evoked device, as described herein, may allow for an accurate, noninvasive, real-time method for the diagnosis, management, and/or alerting of HIE or stroke in a patient.

In some implementations, the EM-evoked device may include a miniature probe for use during early labor (e.g., when cervical dilation is only a few centimeters) to enable detection of a lack of oxygen to the brain (e.g., hypoxia) during labor, and thereby enable rapid response during labor (e.g., to perform a cesarean section, adjust a position of the fetus in the uterus or birth canal, adjust a position of a mother of the fetus, and/or the like). In some implementations, the device may be aligned with the superior sagittal sinus (SSS) of the fetus or newborn to monitor global oxygenation of the brain. In some implementations, the EM-evoked device may include a relatively larger (e.g., relative to a miniature version of the photoacoustic device, NIRS technology, and/or the like) probe for monitoring a brain of a newborn child (e.g., from immediately after birth) to enable greater spatial imaging resolution of brain hemodynamics of the newborn child.

Furthermore, some implementations described herein enable a determination of a probability that a patient may experience a stroke (e.g., due to a clot resulting in reduced flow of oxygenated blood or a hemorrhage disrupting blood circulation). In some implementations, a helmet-type device may be used to measure a parameter associated with oxyhemoglobin saturation and/or hemoglobin concentration (e.g., total hemoglobin concentration, and/or the like) in the brain of the patient to determine the probability of a stroke. For example, before or after delivery, an EM-evoked helmet-type device may be placed over the head of the patient to measure the brain tissue oxyhemoglobin saturation and/or hemoglobin concentration and determine the probability of a stroke according to the measured parameters.

In this way, an EM-evoked device is provided that enables monitoring and detection of HIE and/or a stroke in a patient. In some implementations, the patient may be a fetus and/or newborn child. Accordingly, the EM-evoked device may enable a potential injury to the fetus and/or newborn child to be prevented and/or may enable further injury to the fetus and/or newborn child to be prevented (e.g., by enabling one or more operations to be performed to address the HIE and/or stroke in the patient).

While some implementations are described herein in connection with monitoring and/or detecting HIE and/or a stroke, such implementations may similarly apply and/or be used in other clinical applications, such as detecting internal bleeding, brain death, or other biological conditions or associated biological responses.

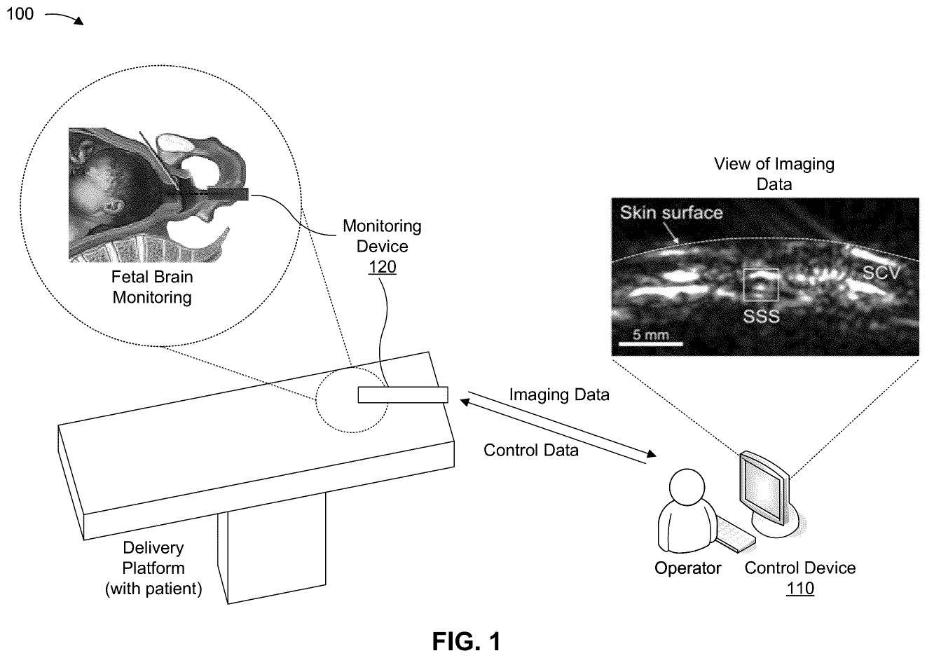

FIG. 1 is a diagram of an example implementation 100 described herein. As shown in FIG. 1, example implementation 100 includes a control device 110 and a monitoring device 120.

Although some implementations described herein are described in terms of monitoring a fetus during labor, some implementations described herein may be used for monitoring a patient before labor (i.e., a fetus) and/or monitoring a patient after labor (e.g., a newborn, an infant, a child, or an adult).

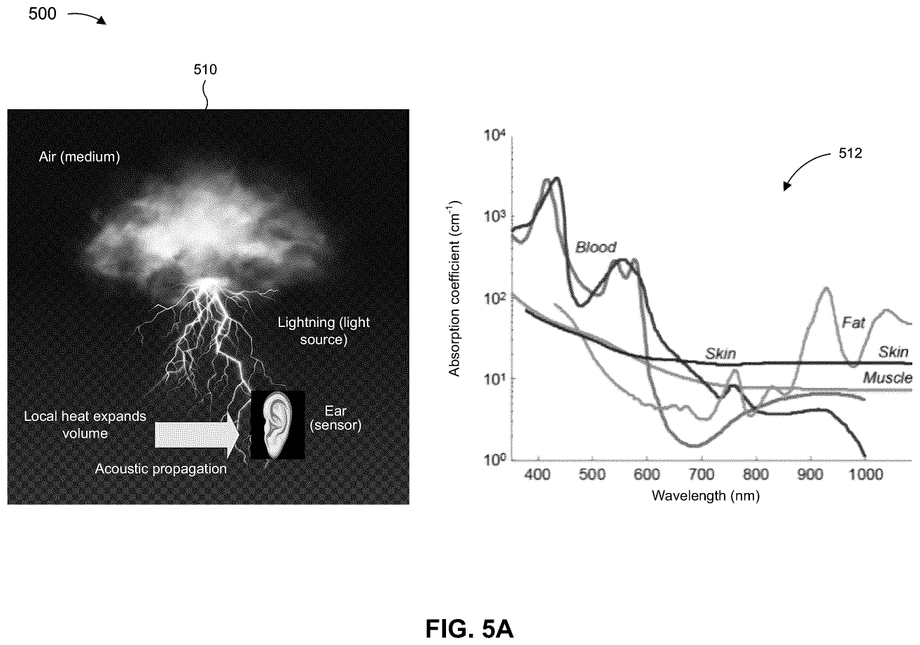

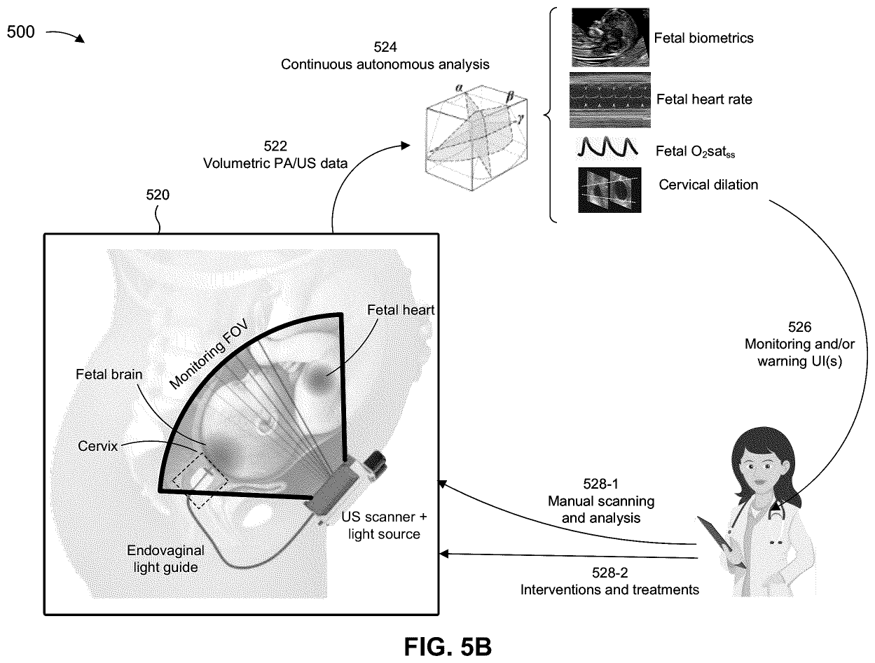

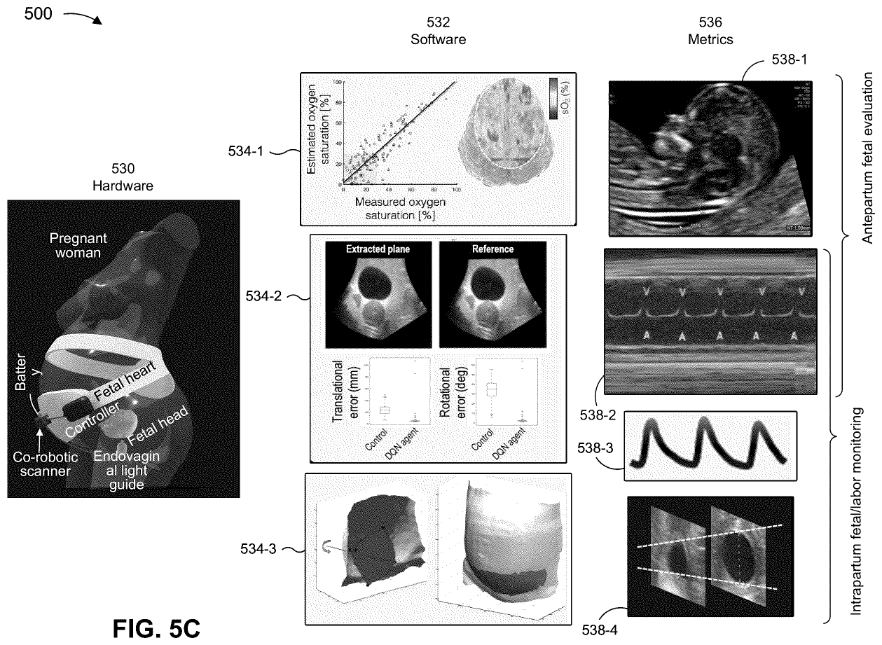

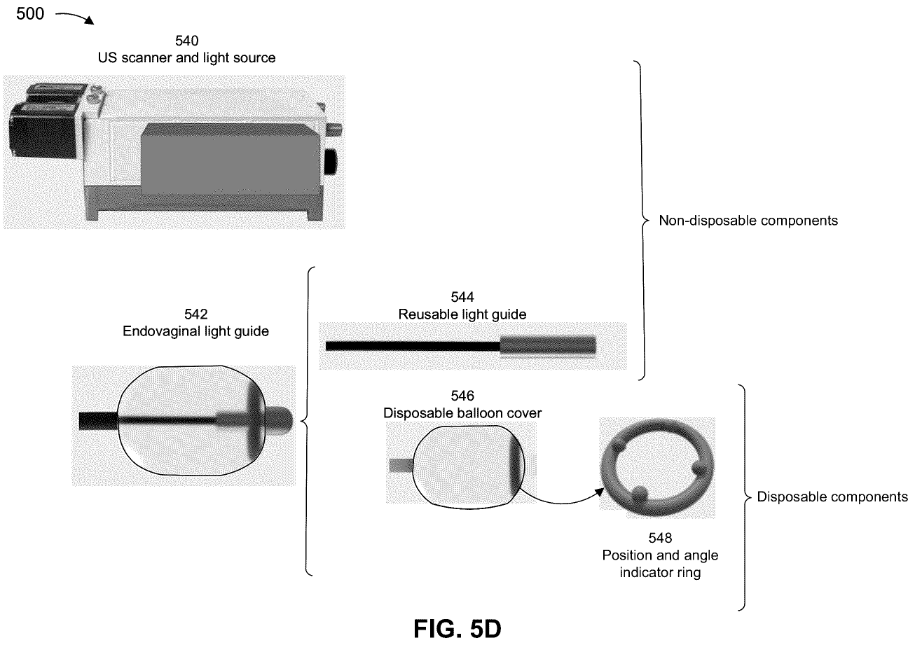

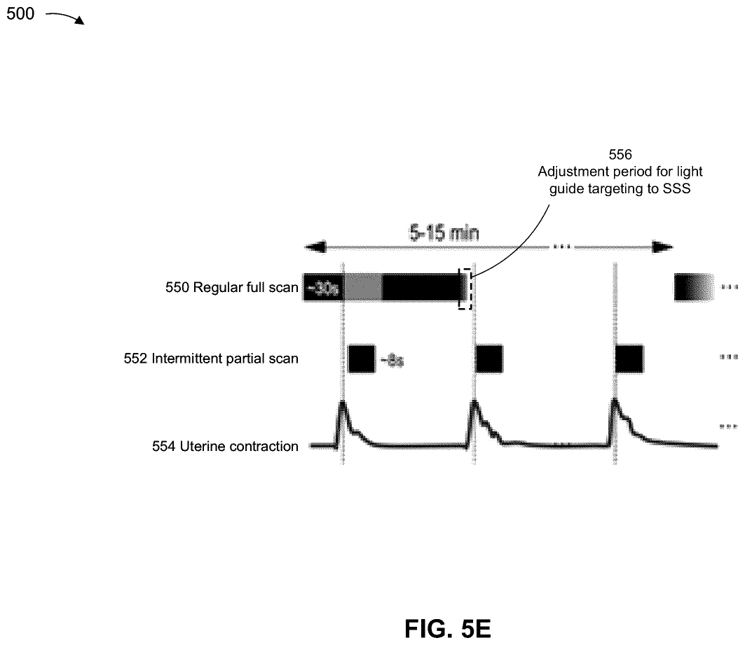

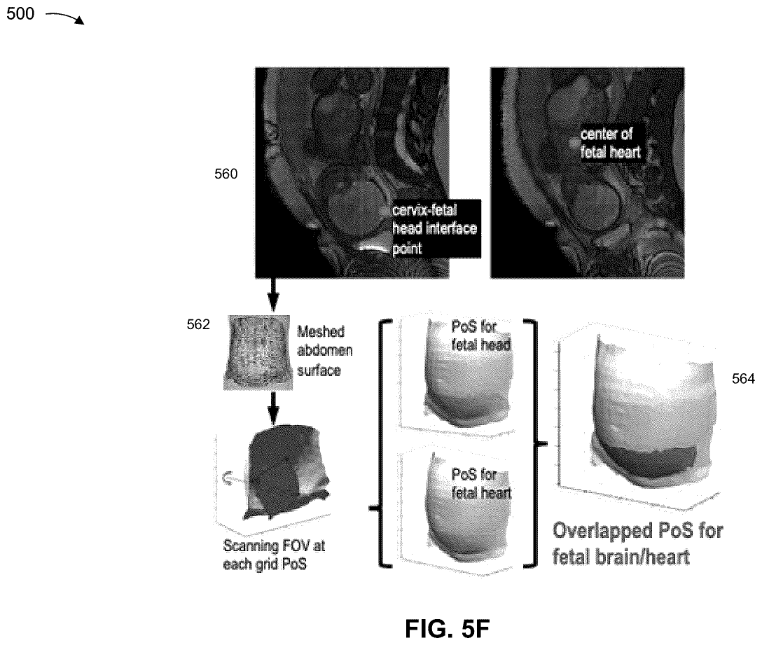

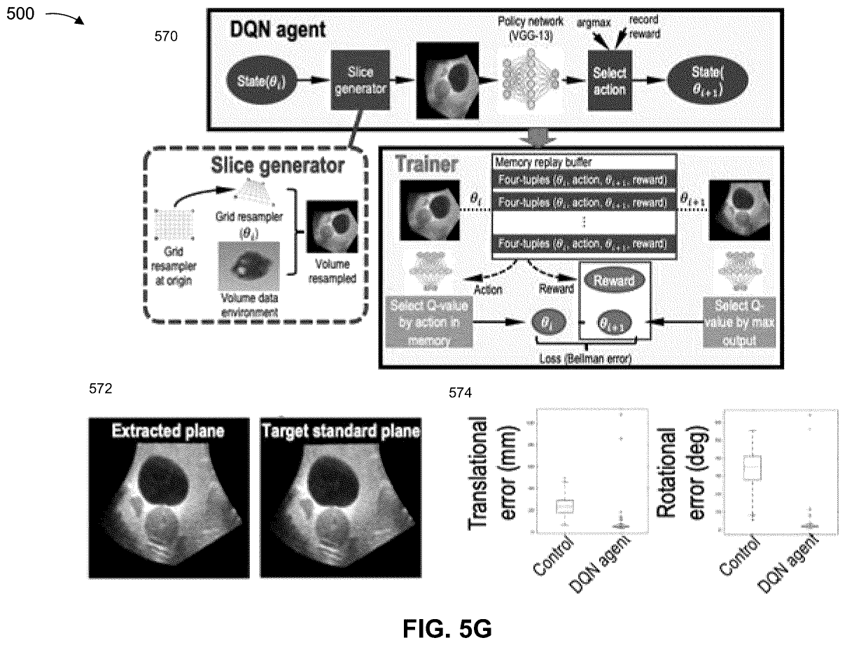

As further shown in FIG. 1, control device 110 may include a monitoring control device with a display device that may be operated by an operator (e.g., an obstetrician, a midwife, a nurse, and/or the like). As described herein, monitoring device 120 may include an EM-evoked device that has an EM component, an ultrasound sensing component, and an ultrasound transmission component. In some implementations, monitoring device 120 may include an electroencephalography (EEG) component. Additionally, or alternatively, the monitoring device 120 may include a wearable photoacoustic and ultrasound device, which includes an ultrasound scanner and a light source within a wearable housing and an endovaginal light guide for guiding light that is emitted by the light source into the birth canal (e.g., as described in more detail below with reference to FIGS. 5A-5G).

In some implementations, control device 110 may provide control data to monitoring device 120. For example, control device 110 may cause monitoring device 120 to perform one or more operations to obtain imaging data of tissue of a patient (shown as a fetus) on a delivery platform, to incite a neural response from tissue of the patient, to measure a parameter associated with the neural response, and/or the like.

In some implementations, control device 110 may cause the EM component of monitoring device 120 to emit EM energy toward tissue (e.g., brain tissue, abdominal tissue, and/or the like) of the patient to cause the EM energy to be absorbed by the tissue. As described herein, when emitting energy (e.g., EM energy, such as light energy, thermal energy, and/or the like), the electromagnetic component may emit the energy in a continuous manner (e.g., in an always-on mode or always-active mode with a minimum threshold magnitude during a designated time period), in a pulsed manner (e.g., alternating from an on-mode to an off-mode according to a particular frequency, time period, schedule, and/or the like), according to one or more modulation techniques (e.g., analog modulation, digital modulation, and/or the like), and/or the like.

The EM component may include an optical component to continuously emit light or emit light pulses (e.g., laser pulses, near infrared (NIR) light pulses, and/or the like) and/or a thermal component to emit thermal energy (e.g., as continuous (or ongoing) microwaves or pulses of microwaves) toward the tissue. In some implementations, control device 110 may cause the ultrasound sensing component of monitoring device 120 to sense acoustic signals generated from absorbers (e.g., molecules, cells, and/or the like that absorb light) in the tissue of the patient that absorbed the energy emitted by the EM component of monitoring device 120. As mentioned above, absorption of the energy causes acoustic signals to be formed as a result of thermoelastic expansion of the tissue. Accordingly, control device 110 may cause the ultrasound sensing component of monitoring device 120 to generate imaging data (e.g., for EM imaging) corresponding to sensing the energy being absorbed by the tissue. The imaging data may be used to generate an image of the tissue based on the sensed energy absorption. In some implementations, the imaging data may correspond to an image stream (e.g., a series of images that can be used to produce a video of the tissue). In some implementations, control device 110 may configure one or more of a frequency associated with a frame rate of the image stream (e.g., a temporal resolution corresponding to how often an image is to be captured), a spatial resolution of images in the image stream (e.g., a high contrast resolution to permit sensing of target molecules in the tissue), and/or the like.

In some implementations, control device 110 may cause an ultrasound transmission component of monitoring device 120 to transmit acoustic energy toward the tissue to cause a biological response from the tissue. The biological response may include a neural response corresponding to neuronal activity involving the firing or non-firing of one or more neurons based on a stimulant. The biological response may correspond to bleeding or other types of biological activity performed by the tissue as a result of the acoustic energy. As described herein, the ultrasound transmission component of monitoring device 120 may interrogate the tissue (e.g., by inciting a neural response in the tissue and capturing an image of the neural response) of the patient to enable the neural response to be analyzed to enable detection of the health status of the tissue. Control device 110 may control the ultrasound transmission component of monitoring device 120 using a control signal, such as a voltage application, an excitation of a laser energy, an excitation of a thermal energy, and/or the like.

Additionally, or alternatively, control device 110 may cause the EEG component to sense electrical activity associated with the tissue. For example, control device 110 may use electrical activity data corresponding to the sensed electrical activity to determine timing associated with the biological response (e.g., based on changes in voltage, current, and/or the like detected in the tissue), determine a status of the tissue, and/or the like. In some implementations, the changes to the electrical activity may be mapped to corresponding moments of a biological response (e.g., a beginning, an intermediate stage, an end, and/or the like). Such timing measurements may be made relative to the transmission of the acoustic energy from the ultrasound transmission component (e.g., to detect whether there was an unexpected delay, to determine a duration of the neural response, and/or the like).

In some implementations, control device 110 may cause the ultrasound sensing component to sense a neural response based on the timing of the neural response determined from the electrical activity data. For example, when control device 110 determines, from the electrical activity data, that the neural response is beginning and/or is about to begin, the control device 110 may cause the ultrasound sensing component of the monitoring device to sense the neural response, as described herein, and/or generate imaging data associated with the neural response. Additionally, or alternatively, when control device 110 determines, from the electrical activity, that the neural response is ending or has ended, control device 110 may cause the ultrasound sensing component to cease sensing the neural response and/or generating imaging data associated with the neural response.

Accordingly, control device 110 may determine, from the electrical activity, when a biological response begins, changes, and/or ends (e.g., to conserve power resources and/or computing resources that might otherwise be wasted attempting to sense, image, and/or analyze a biological response that has not begun or that has ended). In this way, when combined with the imaging data from the ultrasound sensing component, control device 110 may determine a status of the tissue.

As described herein, control device 110 may detect variations in response perturbations in energy demand from transmissions of the ultrasound transmission component and provide information on the health status of the tissue. For example, control device 110 may measure, from images associated with a neural response, any change in oxyhemoglobin saturation, hemoglobin concentration, cytochrome aa3 oxidized state, and/or lipid contents in the tissue. Additionally, or alternatively, the control device may determine timing and/or a status of the tissue based on electrical activity detected by the EEG component. In this way, control device 110 may quantitate one or more parameters associated with the tissue in order to assess the condition of a brain tissue. Correspondingly, the ultrasound transmission component of monitoring device 120 may enable control device 110 to determine the oxygenation and/or hemoglobin concentration of a fetal brain during labor and/or the brain of a child after birth. Accordingly, including the ultrasound transmission component in monitoring device 120 enables monitoring device 120 to incite a neural response based on oxygen demand and/or vascular responses to neuronal activation in the tissue and capture dynamic changes in the brain as a biomarker of the health status of the tissue.

As further shown in example implementation 100 of FIG. 1, monitoring device 120 may enable fetal brain monitoring. For example, monitoring device 120 may be inserted into a birth canal and received within a uterus (e.g., when the cervix is dilated to at least the width of monitoring device 120). Monitoring device 120 may then provide imaging data associated with the fetal brain to control device 110, which may cause an image associated with the imaging data to be displayed via a user interface (e.g., a monitor, a touchscreen, and/or the like) communicatively coupled to control device 110. In this way, based on providing such a visualization, control device 110 reduces a likelihood of complications during and/or after the birth of a child relative to other techniques that do not provide such accurate imaging of oxyhemoglobin saturation and/or corresponding neural responses, as described herein.

In some implementations, control device 110 may process the imaging data to provide contextual information associated with the tissue. For example, control device 110 may perform a pixel-by-pixel, ratio-metric measurement between multi-wavelength photoacoustic or thermoacoustic signals sensed in a field-of-view of monitoring device 120. Control device 110 may measure values of the pixels (e.g., corresponding to an intensity of the pixels) from imaging data to determine a measurement of a parameter associated with a neural response. The measurement may correspond to changes in the values of the pixels over a time period associated with the neural response, which represents a change in one or more parameters (e.g., an oxyhemoglobin saturation, a hemoglobin concentration, a cytochrome aa3 oxidized state, a lipid content, and/or the like) of the tissue. In some implementations, the wavelength may be set for multi-spectral photoacoustic or thermoacoustic sensing, according to which target molecules (e.g., hemoglobin, cytochrome aa3, lipid, and/or the like) are to be measured (sensed) in the tissue. Furthermore, the temporal resolution of the imaging may be configured according to desired measurements of the molecules.

In some implementations, control device 110 may detect a hypoxic condition in the tissue when an EM-evoked oxygen saturation of hemoglobin in an SSS of a fetus and/or child is less than a particular level (e.g., less than 30%). In some implementations, control device 110 may perform a least-mean-square error estimation for a measurement of a particular parameter (e.g., hemoglobin, cytochrome aa3, lipid, and/or the like) of the tissue and/or neural response of the tissue. For example, light absorption can be measured using spectrophotometric measurement of ex vivo tissue samples (e.g., other tissue samples that have been previously analyzed and/or measured using the same technique). Accordingly, an EM-evoked oxygen saturation can be estimated at the SSS using a least-mean-square error estimation between obtained multispectral photoacoustic data associated with photoacoustic imaging of the SSS and known spectrophotometric absorbance of hemoglobin. Accordingly, control device 110 may determine a quantitative indication of a value of a parameter (e.g., oxygen saturation) of the tissue (e.g., in a particular region of the tissue). In some implementations, control device 110 may measure a change in oxyhemoglobin concentration and/or hemoglobin concentration in tissue (e.g., cortical brain tissue) from acoustic estimations of the oxygen saturation and/or hemoglobin concentration over a time period associated with a neural response. Additionally, or alternatively, control device 110 may detect a stroke associated with a brain of fetus and/or child based on acoustic estimations of changes in oxygen saturation and/or hemoglobin concentration, as described herein. In some implementations, thermoacoustic imaging can be used to sense and/or detect deep neuronal activity of the brain and/or biological activity in fat tissue and/or muscle tissue, sense or detect water contents, sense or detect internal bleeding, and/or the like. Other biological tissue providing absorptive contrast may be visualized for differential diagnoses of diseases.

In some implementations, control device 110 may use one or more artificial intelligence techniques (e.g., machine learning, deep learning, and/or the like) associated with processing imaging data (e.g., via pattern recognition, neural networks, heuristics, and/or the like) from monitoring device 120. Accordingly, the one or more artificial intelligence techniques may enable control device 110 to automatically identify, from the imaging data and/or electrical activity data received, a change in saturation of oxyhemoglobin and/or hemoglobin concentration associated with an incited biological response in a particular region of the tissue (e.g., indicative of a stroke in a particular region of the brain), and provide corresponding information to an operator. For example, control device 110, via a user interface, may alert the operator (e.g., through an audible alarm, a visual alarm, a vibrating alarm, and/or the like) when a measured value of a parameter indicates that the patient is likely experiencing a stroke and/or is about to experience a stroke (e.g., when the value satisfies a threshold representative of a stroke being imminent and/or occurring). Based on the alert provided via the user interface, the operator may perform one or more operations to attempt to prevent injury or further injury to the patient.

Accordingly, control device 110 may use a machine learning model to identify a status of the tissue that is being monitored and/or a change to the status of the tissue over a time period associated with the biological response. For example, control device 110 may train the machine learning model based on measuring, from imaging data, one or more parameters associated with identifying the status of the tissue and/or a change in the status of the tissue, such as an oxygen saturation level, a hemoglobin concentration level, a cytochrome aa3 oxidized state (e.g., a level of the cytochrome aa3 oxidized state), an amount of lipids in the tissue, an amount of changes to one or more parameters within a particular time period (e.g., corresponding to a neural response), a type of the tissue, an operation associated with the tissue (e.g., delivering a patient associated with the tissue), a spatial resolution associated with one or more images associated with the imaging data, a temporal resolution associated with one or more images associated with the imaging data, electrical activity associated with the tissue, timing associated with the electrical activity associated with the tissue (which may correspond to timing of the neural response), and/or the like. Control device 110 may train the machine learning model using historical data associated with identifying the status according to the one or more parameters. For example, the historical data may be associated with measuring the one or more parameters from other imaging data associated with one or more other biological responses incited in tissues of one or more other patients. Using the historical data and the one or more parameters as inputs to the machine learning model, control device 110 may identify the status, to permit an operation associated with the patient to correspondingly be performed. Additionally, or alternatively, the machine learning model may consider one or more other metrics of the patient that are being monitored, such as auditory-evoked potential (AEP), blood pressure, pulse amplitude, pulse frequency, peripheral capillary oxygen saturation (SpO2), and/or the like.

Accordingly, control device 110 may automatically and/or objectively determine the status of tissue based on processing imaging data associated with a biological response in the tissue. In some implementations, control device 110 may automatically perform one or more actions associated with a determined status of the tissue according to the imaging data provided by monitoring device 120. For example, control device 110 may determine that the tissue of the patient is hypoxic based on a value of the estimated oxygen saturation, a value associated with an amount of cytochrome aa3, a value associated with lipid contents of the tissue, and/or the like. In such cases, control device 110 may alert, via a user interface, the operator that the patient may be developing HIE, in order to permit the operator to take appropriate action (e.g., perform a cesarean section, adjust a position of the fetus in the uterus and/or birth canal, and/or the like).

Accordingly, as described herein, monitoring device 120 enables monitoring of tissue of a patient through photoacoustic and/or thermoacoustic sensing and modulation. An EM component and an ultrasound sensing component of monitoring device 120 may provide a multi-spectral acoustic sensing device for noninvasive biological function estimation, and an ultrasound transmission component of monitoring device 120 may provide an ultrasound modulation element to interrogate a health of a tissue of a patient, including the health of muscle tissue, a particular organ tissue (e.g., brain tissue), and/or the like. Furthermore, control device 110 may analyze imaging data received from monitoring device 120 to detect a particular status of the tissue. Accordingly, in some implementations, monitoring device 120 and/or control device 110 may enable rapid identification of any development of cerebral hypoxia in a patient (e.g., a fetus during labor and/or a child after labor), so that appropriate care can be administered to the patient before potential arterial hypotension and/or metabolic acidosis become sufficiently severe to cause permanent damage.

As indicated above, FIG. 1 is provided as an example. Other examples may differ from what is described with regard to FIG. 1.

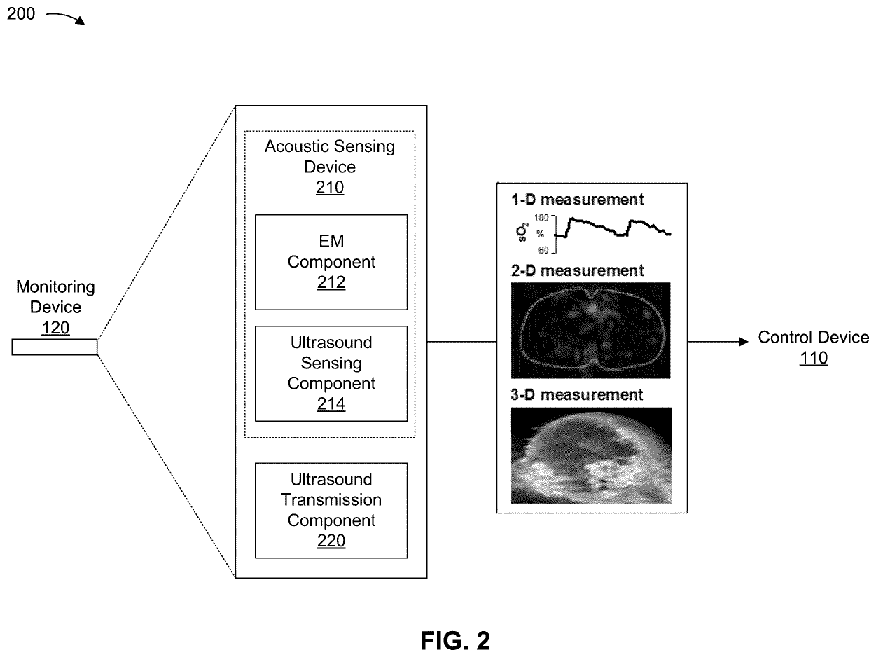

FIG. 2 is a diagram of an example implementation 200 described herein. FIG. 2 depicts a monitoring device 120 capable of providing imaging data to a control device (e.g., control device 110). Monitoring device 120 of example implementation 200 may correspond to monitoring device 120 of FIG. 1. Monitoring device 120 of FIG. 2 includes an acoustic sensing device 210, which includes an EM component 212, an ultrasound sensing component 214, and an ultrasound transmission component 220.

EM component 212 may include one or more components to emit EM energy. For example, EM component 212 may include an optical component with a light emitter to emit light toward tissue of a patient in order to cause the light to be absorbed by the tissue. EM component 212 may include one or more optics (e.g., one or more lenses, one or more optical fiber bundles, such as bifurcated optical fiber bundles, and/or the like) and a light source (e.g., an emitter element, such as a laser emitting diode). Similarly, EM component 212 may include a thermal component that includes a microwave generator and/or microwave emitter to generate and/or emit EM energy as thermal microwave energy (which may be referred to herein as microwaves) toward tissue of a patient in order to cause the EM energy to be absorbed by the tissue. In some implementations, EM component 212 may be tunable to emit pulsed EM energy (e.g., pulsed light, such as pulsed NIR light in the range of 680-2400 nanometers (nm)) with a nanosecond-scale pulse duration and a repetition rate over tens of hertz (Hz). Accordingly, control device 110 may tune EM component 212 of monitoring device 120 to make real-time measurements of target molecules that are to be photoacoustically or thermoacoustically sensed according to a desired spatial resolution, a desired temporal resolution, and/or the like to permit control device 110 to estimate oxyhemoglobin saturation, hemoglobin concentration, cytochrome aa3 oxidized state, lipid contents, and/or the like in the tissue.

Ultrasound sensing component 214 is configured to sense absorption of the energy emitted by EM component 212. As described herein, the energy (e.g., light waves, microwaves, and/or the like) may be absorbed over time during a biological response (e.g., a biological response incited by ultrasound transmission component 220). Accordingly, ultrasound sensing component 214 may be configured to sense the biological response based on the energy being absorbed in the tissue during the biological response. Furthermore, ultrasound sensing component 214 may generate imaging data associated with the biological response to permit a status of the tissue to be determined (e.g., from the imaging data).

Ultrasound sensing component 214 may include one or more piezoelectric elements. In some implementations, the number of elements may provide a corresponding dimensional measurement. For example, a zero-dimensional configuration (e.g., a single element) may provide a one-dimensional measurement (e.g., time sequence of a sensed acoustic signal, as shown in the 1-D measurement), a one-dimensional configuration (e.g., a linear array of elements) may provide a two-dimensional measurement (e.g., a cross-sectional acoustic image, as shown the 2-D measurement), and a two-dimensional configuration may produce a volumetric acoustic image (e.g., a three-dimensional image, as shown in the 3-D measurement). Accordingly, ultrasound sensing component 214 may be suitably configured as described herein to provide a measurement and/or image associated with a biological response in tissue to permit the health status of the tissue to be determined.

Ultrasound transmission component 220 of monitoring device 120 may be an ultrasound modulation component (e.g., an ultrasound neuromodulation component) that includes a piezoelectric element and/or a thermoelastic element that can provide sufficient acoustic power that satisfies a threshold to cause a biological response, such as a neuronal depolarization in the tissue (e.g., in the brain). In some implementations, ultrasound transmission component 220 may include one or more ultrasound emitting elements according to a desired flexibility with respect to delivering the energy. For example, a single ultrasound element may not have flexibility in that the single ultrasound element provides the acoustic energy in a fixed direction and/or from a fixed position (e.g., via an acoustic lens associated with the single ultrasound element). On the other hand, an array-based ultrasound element configuration (which includes a plurality of ultrasound elements) may enable ultrasound transmission component 220 to focus transmissions in a plurality of different directions and/or on a plurality of different positions of a three-dimensional space (e.g., within a specific brain lobe, within a cortical region of the brain, and/or the like). For example, the EM-evoked sensing and neuromodulation probe components may include a 2-D array of ultrasound sensing elements within a 3-D spatial domain to permit monitoring device 120 to monitor volumetric hemodynamics in a brain.

As described herein, ultrasound sensing component 214 of the 2-D array can be implemented in any suitable configuration with any suitable number of elements or types of elements (e.g., one or more crossed linear array configurations for cost-effectiveness, a uniformly distributed element configuration for optimal spatial resolution, a randomly distributed element configuration for better suppression of side lobe, and/or the like). Furthermore, a configuration of an optical component of EM component 212 may include an optical fiber bundle within the 2-D array (e.g., for relatively deep light penetration), an optical fiber bundle that is uniformly distributed at a transmission end of the probe adjacent ultrasound sensing component 214 and/or ultrasound transmission component 220 (e.g., to configure a relatively wide field of view), an optical fiber bundle randomly distributed at the probe end adjacent ultrasound sensing component 214 and/or ultrasound transmission component 220 (e.g., to allow for relative uniformity).

In some implementations, ultrasound transmission component 220 of monitoring device 120 may be configured to transmit sub-thermal ultrasound energy (e.g., to satisfy certain laws and/or regulations associated with transmitting ultrasound energy toward tissue of a patient). In some implementations, ultrasound transmission component 220 may be configured to increase an adenosine triphosphate (ATP) demand in the tissue, change a cytochrome aa3 oxidation state of the tissue, and/or cause oxyhemoglobin saturation in the tissue. Therefore, ultrasound transmission component 220 of monitoring device 120 may enable noninvasive monitoring of dynamic biological responses (e.g., neural responses, oxygenation responses, bleeding, and/or the like) in tissue to perturbations in energy demand via modulation and allow for the portrayal of a transition to a pathological state (e.g. an increasing extent of hypoxia).

In some implementations, a configuration of acoustic sensing device 210 and ultrasound transmission component 220 of monitoring device 120 may be based on a desired imaging width, a desired imaging aspect ratio, a desired size, and/or the like. For example, monitoring of an intrapartum fetal brain may involve monitoring device 120 having a relatively compact probe size (a linear probe with a width of less than six millimeters (mm) to enable the monitoring device 120 to be received in a uterus during labor) and a particular monitoring width in field-of-view to monitor the fetal brain from an end of monitoring device 120. On the other hand, one or more monitoring devices 120 that are relatively larger can be spatially arranged around the head of a child after birth to enable multiple fields of view.

In this way, monitoring device 120 may include EM component 212, ultrasound sensing component 214, and ultrasound transmission component 220 to permit monitoring device 120 to incite a biological response associated with the tissue, provide information associated with the biological response to control device 110, and thus enable a particular action associated with the patient to be taken (e.g., by an operator associated with control device 110).

As indicated above, FIG. 2 is provided as an example. Other examples may differ from what is described with regard to FIG. 2.

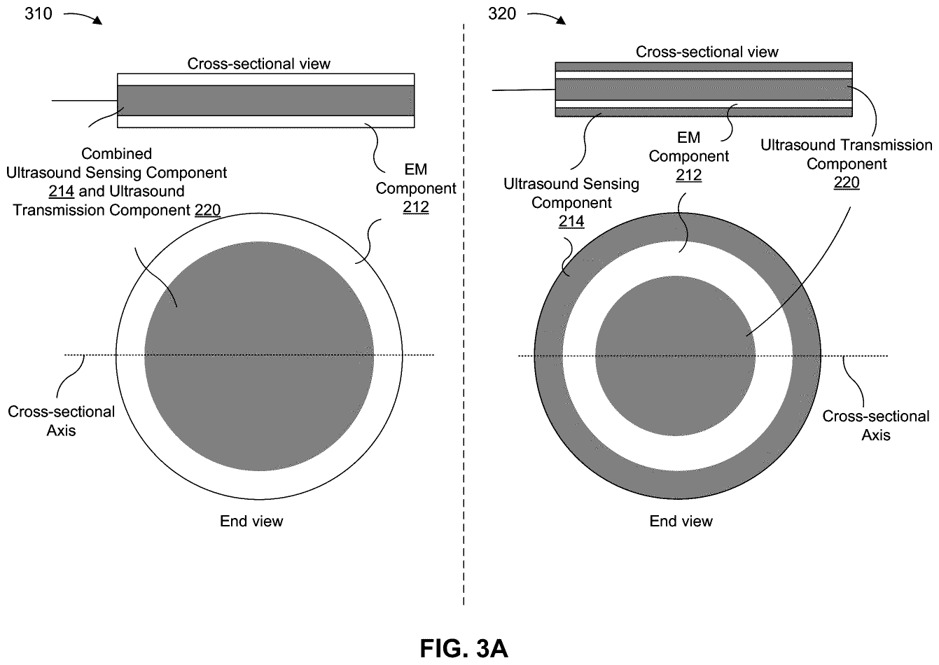

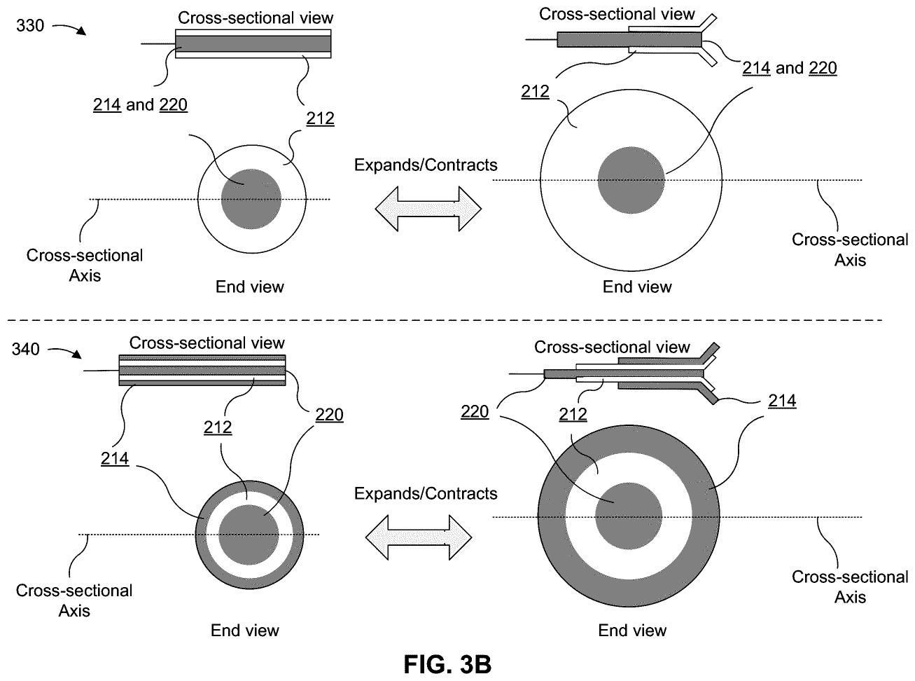

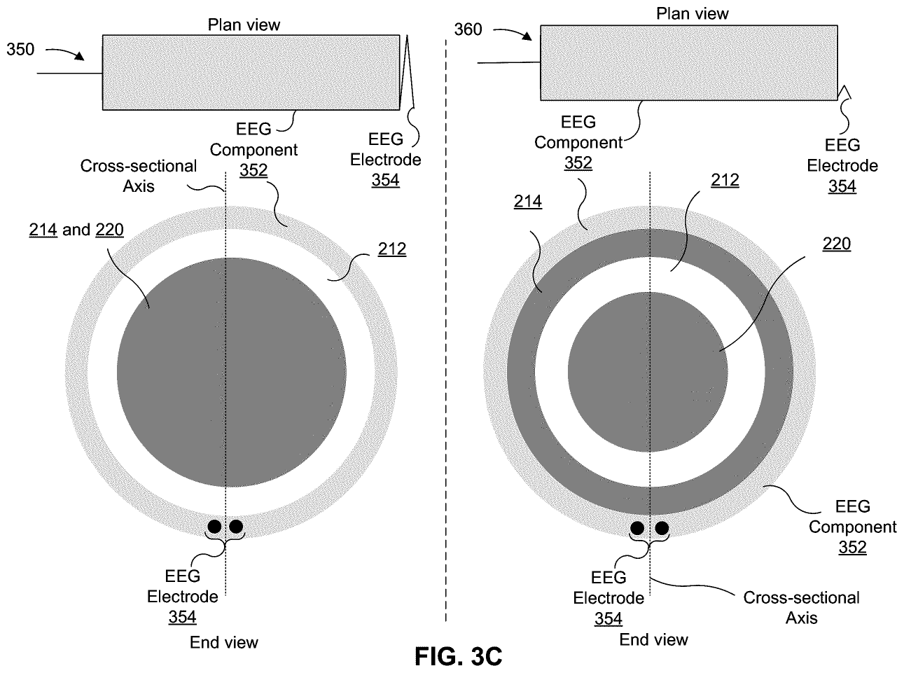

FIGS. 3A-3C are diagrams of example implementations of an EM-evoked device described herein. In FIGS. 3A-3C, an end view and a cross-sectional view (FIGS. 3A and 3B) or an end view and a plan view (FIG. 3C) of example implementations of a monitoring device, such as monitoring device 120 of FIGS. 1 and/or 2, are shown. In FIGS. 3A-3C, the example implementations are configurations of the monitoring device as a linear probe. In some implementations, one or more additional components may be included (e.g., a finger grip, an adhesive element that can be adhered to fetal scalp (e.g., via a gel), and/or the like). Additionally, or alternatively, the EM-evoked device of the example implementations of FIGS. 3A-3C may include a housing. The housing may be a transparent and/or translucent material that enables light to be emitted through the housing toward the tissue.

As shown in FIG. 3A, and by reference number 310, EM component 212 may be or may include a tubular EM element. For example, EM component 212 may include a tube of optics, such as an optical fiber bundle in the shape of a tube. Additionally, or alternatively, EM component 212 may include a tube shaped microwave emitter (or microwave emitting elements) that emits thermal EM energy in microwaves.

Furthermore, as shown by reference number 310, ultrasound sensing component 214 and ultrasound transmission component 220 may be a same element. For example, ultrasound sensing component 214 and ultrasound transmission component 220 may be implemented via a same piezoelectric element or a same thermoelastic element. Accordingly, as shown by reference number 310, an element to perform the operations of ultrasound sensing component 214 and ultrasound transmission component 220, as described herein, may be situated coaxially within tubular EM component 212. In some implementations, the configuration of EM component 212 and the combined ultrasound sensing component 214 and ultrasound transmission component 220 may be inverted. For example, an element to perform the operations of ultrasound sensing component 214 and ultrasound transmission component 220 may be a tubular element (e.g., a tubular piezoelectric element or a tubular thermoelastic element). In such a case, EM component 212 may be situated coaxially within the tubular element. Furthermore, the tubular element may be translucent in order to permit light from EM component 212 to be emitted through the tubular element.

Accordingly, by combining the functionality of ultrasound sensing component 214 and ultrasound transmission component 220 into a same element, the monitoring device associated with reference number 310 has a compact and efficient probe design (e.g., because two separate elements may not need to be configured to implement ultrasound sensing component 214 and ultrasound transmission component 220).

As further shown in FIG. 3A, and by reference number 320, EM component 212 may be a tubular optical element that is situated coaxially between ultrasound sensing component 214 and ultrasound transmission component 220. For example, as shown by reference number 320, ultrasound sensing component 214 and ultrasound transmission component 220 may be implemented via separate elements, where ultrasound sensing component 214 is a tubular element and ultrasound transmission component 220 is a cylindrical element. Accordingly, ultrasound transmission component 220 may be situated coaxially within EM component 212, and EM component 212 may be situated coaxially within ultrasound sensing component 214. In such cases, ultrasound transmission component 220, being centered within the probe, may enable centered interrogation of a biological function (e.g., to incite the biological response), while ultrasound sensing component 214 may be evenly distributed for uniform photoacoustic generation in a relatively wide field-of-view.

In some implementations, one or more ratios in the areas of EM component 212, ultrasound sensing component 214, and ultrasound transmission component 220 (as viewed from the end view) may be configurable according to desired efficiencies between photoacoustic or thermoacoustic monitoring and ultrasound modulation. For example, for a given area for EM component 212 and ultrasound sensing component 214, the ratio between EM component 212 and ultrasound sensing component 214 may correspond to a spatial resolution, sensing sensitivity, and/or sensing uniformity in a given field-of-view. Further, the given area for ultrasound transmission component 220 may correspond to a transmittance and spatial resolution of modulation with a transmitting efficiency based on a type of material of ultrasound transmission component 220 (e.g., a type of piezoelectric material and/or thermoelastic material).

In some implementations, ultrasound sensing component 214 may be a cylindrical element and ultrasound transmission component 220 may be a tubular element. In such cases, ultrasound sensing component 214 may be situated coaxially within EM component 212, and EM component 212 may be situated coaxially within ultrasound transmission component 220.

In this way, monitoring device 120 can be configured as a linear probe with an EM component 212, an ultrasound sensing component 214, and an ultrasound transmission component 220, as described herein. Such a linear probe may have a length that is substantially longer than the width (e.g., at least twice the width). Accordingly, EM component 212, ultrasound sensing component 214, and/or ultrasound transmission component 220 may be longitudinally situated within the probe.

In FIG. 3B, example implementations are shown to permit a monitoring device (e.g., the monitoring device 120 of FIGS. 1 and/or 2) to have an expandable or contractible field-of-view for photoacoustic or thermoacoustic sensing and/or imaging. As shown in FIG. 3B, and by reference number 330, a dimension (e.g., an external radius) of EM component 212 may expand and/or contract relative to a combined ultrasound sensing component 214 and ultrasound transmission component 220. For example, the monitoring device may be inserted into the uterus in a compact configuration, and, after the monitoring device is inserted within the uterus, the end (e.g., a transmission end corresponding to the end from which the light may be emitted and/or an ultrasound signal may be emitted) of the monitoring device may expand to widen the field-of-view of the monitoring device. The field-of-view may be widened so that the light may be emitted across a wider area. Accordingly, despite the probe having a compact diameter to permit insertion in the uterus during labor, a wide field-of-view can be achieved by configuring EM component 212 to expand or contract. As further shown in FIG. 3B, and by reference number 340, a tubular EM component 212 and a tubular ultrasound sensing component 214 can similarly be configured to expand and/or contract.

Accordingly, as shown in FIGS. 3A and 3B, EM component 212, ultrasound sensing component 214, and ultrasound transmission component 220 may be configured within a probe. In some implementations, EM component 212, ultrasound sensing component 214, and ultrasound transmission component 220 may be configured as one or more separate elements of monitoring device 120 (e.g., that are not integrated into a same housing). For example, EM component 212 may be an optical fiber bundle or microwave emitter that is a separate element from a piezoelectric element that is used to implement ultrasound sensing component 214 and ultrasound transmission component 220. Additionally, or alternatively, EM component 212 may be a component that is a separate element from both a first piezoelectric element that is used to implement ultrasound sensing component 214 and a second piezoelectric element, which is separate from the first piezoelectric element, that is used to implement ultrasound transmission component 220. However, in such cases, the separate components may be configured to correspondingly monitor (e.g., as controlled by control device 110) the tissue and provide information to control device 110, as described herein.

In FIG. 3C, example implementations are shown to permit a monitoring device (e.g., the monitoring device 120 of FIGS. 1 and/or 2) to include an EEG component 352 with an EEG electrode 354. As shown in FIG. 3C, and by reference number 350 and reference number 360, EEG component 352 may be configured as an external component (e.g., within a tubular housing of the probe) of the monitoring device, such that EM component 212, ultrasound sensing component 214, and/or ultrasound transmission component 220 are coaxially configured within EEG component 352. The EEG component includes an EEG electrode 354 that is configured to detect and/or sense electrical activity within the tissue. For example, the electrode may detect a voltage and/or current within the tissue via leads of EEG electrode 354. In some implementations, EEG component 352 may include a plurality of EEG electrodes 354.

In some implementations, EEG electrode 354 of EEG component 352 may be implemented via a spiral bipolar electrode. Accordingly, as shown by reference number 350, in the cross-sectional view, the spiral bipolar electrode may have a diameter that is the same or similar (e.g., within a tolerance) to an overall diameter of the probe. Additionally, or alternatively, as shown by reference number, in the cross-sectional view, the spiral bipolar electrode may have a diameter that is less than the overall diameter of the probe (e.g., a diameter corresponding to a thickness of EEG component 352). In some implementations, EEG component 352 may be a shaped such that EEG component 352 can be placed adjacent (e.g. parallel to) optic component 212 in the implementation associated with reference number 350 or adjacent to ultrasound sensing component 214 in the implementation associated with reference number 360.

As described herein, EEG component 352 is configured to sense and/or detect electrical activity associated with the tissue. For example, EEG component 352 may measure electrical activity associated with a neural response of the brain that was incited by acoustic energy from ultrasound transmission component 220. In such cases, EEG electrode 354 may detect timing associated with the neural response. For example, when there are one or more changes in the electrical activity (e.g., a change that corresponds to a particular pattern) of the brain, the EEG electrode 354 may capture measurements corresponding to a beginning of the neural response, an intermediate stage of the neural response, and/or an end of the neural response. In such cases, the timing may be determined relative to the transmission of the acoustic energy by ultrasound transmission component 220 to determine a status of the brain. For example, control device 110 may determine from the electrical activity and imaging data received from ultrasound sensing component 214 whether the response was representative of a normal response, indicating that the brain is not experiencing stress or injury, and/or an abnormal response, indicating that the brain is experiencing stress or injury.

Accordingly, as shown in FIG. 3C, EM component 212, ultrasound sensing component 214, ultrasound transmission component 220, and EEG component 352 may be configured within a probe. In some implementations, EM component 212, ultrasound sensing component 214, ultrasound transmission component 220, and/or EEG component 352 may be configured as one or more separate elements of monitoring device 120 (e.g., that are not integrated into a same housing). For example, EM component 212 may be an optical fiber bundle or microwave emitter that is a separate element from one or more piezoelectric elements that are used to implement ultrasound sensing component 214 and/or ultrasound transmission component 220 and an electrode element that may be used to implement EEG component 352.

As indicated above, FIGS. 3A-3C are provided as an example. Other examples may differ from what is described with regard to FIGS. 3A-3C. For example, although the example implementations of FIGS. 3A-3C are shown to be circular in shape, a monitoring device, as described herein, may be any other suitable shape, such as elliptical, rectangular, trapezoidal, triangular, and/or the like. Further, a device, such as a helmet-type device that is configured to fit over a head of a patient, a structured device with a frame shaped to fit a particular body part (e.g., an abdomen, a rib cage, a torso, a limb, and/or the like), and/or the like may be fit to include a plurality of one or more of the components of monitoring device 120 in examples FIGS. 3A-3C. In such cases, the plurality of components may be controlled synchronously (e.g., by control device 110) to monitor the patient as the patient is wearing the device or fit with the device (e.g., when the patient is a newborn or no longer in the womb).

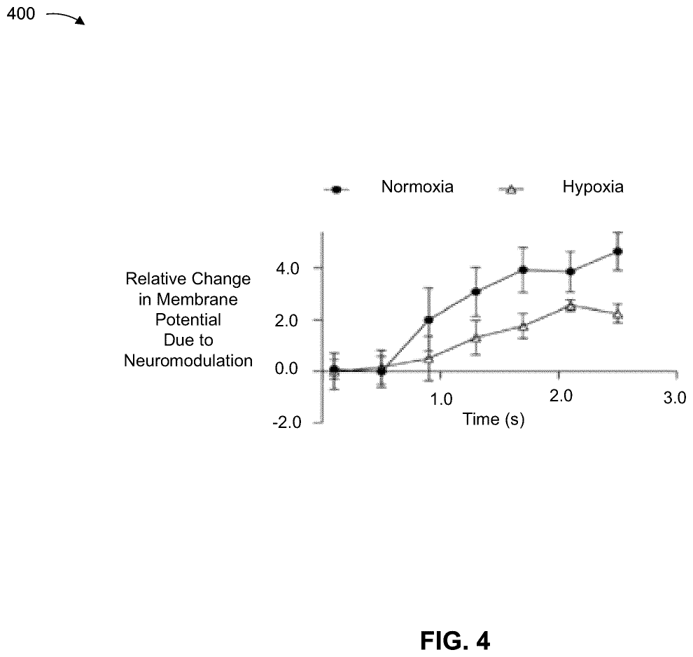

FIG. 4 is a diagram regarding an example implementation 400 described herein. Example implementation 400 includes a graph of time versus a relative change in membrane potential due to neuromodulation, which can be used to indicate oxygen concentration in a tissue of a patient. Accordingly, the graph may represent a normoxic neural response (indicating a standard, healthy response) and a hypoxic neural response (indicating development of HIE) to acoustic energy transmitted by ultrasound transmission component 220, as described herein.

As shown in the graph of example implementation 400, an ultrasound-induced stimulation of a neural response can be used to differentiate between neurons in a normoxic condition or hypoxic condition. As shown in FIG. 4, just after 2.0 seconds in time, there is a decrease in the ultrasound-evoked membrane potential in hypoxic conditions. Therefore, as described herein, a neural response to ultrasound neuromodulation can enable assessment of oxygenation of the tissue, and thus enable assessment of whether the tissue may be developing pathological hypoxic conditions.

As indicated above, FIG. 4 is provided as an example. Other examples may differ from what is described with regard to FIG. 4.

FIGS. 5A-5G are diagrams of example implementations 500 of a wearable ultrasound and photoacoustic device for fetal and/or labor monitoring.

More particularly, as described herein, brain damage in the perinatal period can result in devastating life-long disabilities with significant impacts on society and the families of the injured children. The incidence is gestational age-dependent with neonatal encephalopathy occurring in one to three out of every 1000 term infants, and with cerebral palsy affecting 6-9% of babies born at less than 32 weeks and as many as 28% of babies born at less than 26 weeks. A large fraction of these babies are thought to have experienced peripartum hypoxia-ischemia (HI). Since the early 1970s, the cornerstone of modern intrapartum obstetric practice has been electronic fetal heart rate monitoring (EFM) as a screening tool to identify fetuses that are at risk of developing HIE. EFM is one of the most commonly performed monitoring procedures in medicine, being used in 85% of the 4 million babies born in the United States every year. Since the introduction of EFM, the incidence of cesarean deliveries in the United States has increased from 5% to more than 30% of all deliveries, largely based on the imprecise diagnosis of non-reassuring fetal status. However, during this period, the incidence of cerebral palsy has remained largely unchanged, and in countries with a broad range of cesarean rates from 7-30%, the incidence of cerebral palsy in babies with a birth weight above 2500 grams falls in a very narrow range of 1.1-1.3 per 1000 births. These statistics show that a large number of pregnancies are being delivered by cesarean for suspected HIE despite the EFM abnormalities potentially being false positives. Many studies have confirmed the high false positive rate of EFM abnormalities in detecting brain injury, which have been shown to be as high as 99.8%. Because of the increased maternal morbidity associated with high cesarean delivery rates, a large part of which may be due to cesareans performed for non-reassuring fetal heart rate abnormalities and the lack of a decrease in long-term neurologic morbidity in children when pregnancies are monitored with EFM, the overall value and/or benefit of EFM is uncertain.

In addition to the inability to identify fetuses with HIE during labor, identifying babies that have suffered brain injury in the period just after birth is very difficult. The decision as to whether a newborn has HIE is typically made based on a clinical exam, which is subjective, and based on arterial potential of hydrogen (pH), also referred to as acidity or basicity. Hypotonia after birth is commonly used to identify clinical HIE, but many factors other than HI can be responsible for hypotonia. Accordingly, the Apgar score, usually given at one minute and five minutes after birth, is an excellent tool to assess the need for resuscitation. However, the Apgar score correlates poorly with long-term neurodevelopmental outcome. NIRS is an optical modality to measure tissue oxygen (O₂) saturation, but NIRS cannot distinguish arterial and venous compartments due to low spatial resolution, resulting in low clinical sensitivity of ˜50%. Cranial ultrasound (US), performed shortly after birth, is good at detecting intracranial bleeding, but cannot detect HIE. MRI is useful only after edema forms and cells die, and is usually not performed until 7-10 days of life when the neonate is stable for transport. MRI also requires the use of MRI-compatible isolates and the complete absence of any metal with the neonate. Whole-body hypothermia has become the standard of care for treating neonatal HIE. However, to be effective, whole-body hypothermia therapy must be initiated within six hours of birth, which shows the importance of this critical period just after birth. Potential therapies, such as different cooling regimens, xenon gas, and erythropoietin, are being investigated but also will likely require early intervention. Currently, there is no robust way to rapidly and noninvasively identify neonates at risk for developing brain damage. Reliable detection of HI during labor would permit decisions to be made for rapid delivery and thereby shorten the interval between HI and initiation of hypothermia.

Accordingly, a clinical need exists for a device that can identify fetuses at risk for HIE at different stages of labor, as indicated by cervical effacement and dilation, among other factors. To address this need, some implementations described herein relate to a safe, rapid, noninvasive, inexpensive, and easy-to-use intrapartum fetal brain monitor that also informs the state of cervical dilation while providing traditional fetal heart rate data. The device described herein may integrate photoacoustic and ultrasound modalities to noninvasively identify, in real-time, whether a fetus is at risk of developing HIE during labor and/or at risk of failure of labor progression.

For example, as described herein, photoacoustic monitoring has been used to assess brain oxygenation in animals and adults and is based on the photoacoustic effect that sound waves can be generated through the absorption of modulated light. For example, as shown by reference number 510 in FIG. 5A, light that is generated by a light source (e.g., lightning) may travel through a medium (e.g., air), which creates local heat that expands a volume of the medium that the light source travels through. In this way, the expanded volume that is created by the propagation of the light creates an acoustic (e.g., sound wave propagation) that can then be detected by an acoustic sensor. For example, photoacoustic monitoring can provide rich optical contrast at an acoustic spatial resolution (e.g., approximately hundreds of micrometers (μm)) and acoustic penetration depth in biological tissue (e.g., several centimeters). In photoacoustic imaging, safe pulsed light at a specific wavelength is emitted, and the pulsed light is absorbed by a chromophore such as hemoglobin, thereby generating an acoustic pressure that corresponds with the light absorbance of the target region (e.g., a fetal brain or fetal heart). The acoustic pressure may propagate through biological tissue such that the acoustic pressure can be measured by an external ultrasound transducer. In this way, spectroscopic quantification of oxygenated hemoglobin (HbO₂) may be used to monitor fetal brain oxygenation, which may provide a direct and early indicator of brain injury (e.g., due to HIE and/or stroke). Furthermore, the prognostic contrast can be overlaid with ultrasound imaging to deliver an anatomical context in the field-of-view (FOV).

Due to its hybrid nature, photoacoustic monitoring combines highly attractive features attributed to both light and sound, including rich contrast and high versatility in sensing diverse biological targets, excellent spatial resolution not compromised by light scattering, and a relatively low cost of implementation. For example, as shown by reference number 512, different light wavelengths have been shown to produce different absorption coefficients for different biological targets, such as blood, skin, fat, and muscle. In the biomedical diagnostic field, photoacoustic imaging has been highlighted as a useful hybrid modality, which can provide the molecular contrast of light absorbance with sufficient acoustic penetration depth (e.g., several centimeters) and spatial resolution (e.g., 800 micrometers (μm)). In photoacoustic imaging, radio-frequency (RF) acoustic pressure is generated depending on the light absorbance and thermo-elastic property of a target when the light energy at a specific wavelength is delivered to a target. The generated acoustic pressure propagates through biological tissue and is sensed by an ultrasound transducer. Near-infrared range (NIR, approximately 700-900 nanometers (nm)) has been a good optical window to deliver more optical energy safely to deep tissue. Using this physical mechanism and spectral range, clinical applications have been proposed such as melanoma detection, ocular imaging, screening for cancer metastasis, early cancer indicator imaging, and tumor characterization. Furthermore, several strategies are being pursued to image the brain in various scales from rodent to non-human primate animals. In particular, photoacoustic imaging is capable of transcranial imaging a monkey brain using high-power pulsed laser illumination at 1064 nm and 630 nm, respectively. Importantly, the use of the photoacoustic effect can be used in developing surgical guidance, catheter and tool tracking for interventional guidance, molecular imaging of aggressive tumors, and large scale recording of brain electrical activities in mice and piglets. Specifically, cerebral venous oxygenation can be quantified using transcranial photoacoustic imaging in piglets with closed fontanelles.

Accordingly, some implementations described herein may use photoacoustic and/or ultrasound monitoring techniques to enable non-invasive intrapartum detection of dangerously low cerebral venous oxyhemoglobin saturation at superior sagittal sinus (O₂Satₛₛ), abnormal fetal heart rate, and/or failure of labor progress using a wearable spectroscopic photoacoustic and ultrasound monitoring device that gives freedom of movement for the laboring patient, associated with decreased pain, improved quality of uterine contractions, monitoring of fetal descent, and improved maternal-fetal oxygenation. Each output will be co-registered to uterine contraction monitoring by adopting a sensor from the conventional EFM. The device will help caregivers monitor the fetal status faster and more accurately for possible emergency delivery and postdelivery treatments. For example, as shown by reference number 520 in FIG. 5B, the device may include a light-guiding component, shown as an endovaginal light guide, to guide light energy toward tissue (e.g., a fetal brain and/or a fetal heart) to cause the light energy to be absorbed by the tissue. In addition, the device may include an external component, to be worn on an exterior of a body of a patient (e.g., a pregnant woman), where the external component may include an ultrasound scanner and a light source. For example, in some implementations, the ultrasound scanner may include an ultrasound transmission component to transmit acoustic energy toward the tissue to cause a biological response from the tissue and a sensing component to perform one or more of ultrasound or photoacoustic imaging to sense the biological response from the tissue and permit a status of the tissue to be determined, wherein the biological response is sensed based on the light energy absorbed by the tissue during the biological response caused by the acoustic energy transmitted toward the tissue. In this way, as shown by reference number 522, the ultrasound scanner may collect volumetric photoacoustic and/or ultrasound data, which may be provided to a control device. As shown by reference number 524, the control device may perform continuous autonomous analysis based on the volumetric photoacoustic and/or ultrasound data, such as identifying one or more fetal biometrics, a fetal heart rate, a fetal oxygen saturation, and/or a cervical dilation, among other examples. Accordingly, as shown by reference number 526, information related to the photoacoustic and/or ultrasound monitoring, including one or more warning user interfaces (e.g., when the fetal biometrics, fetal heart rate, fetal oxygen saturation, and/or cervical dilation indicate a risk of HIE, failure of labor progression, or other risk factors), may be presented to one or more clinicians. As further shown by reference number 528-1, the clinician(s) may then perform manual scanning and analysis (e.g., repositioning the device to collect more targeted metrics or further data). Additionally, or alternatively, as shown by reference number 528-2, one or more interventions or treatments (e.g., caesarean delivery or treatments to prevent or mitigate cerebral palsy) may be initiated based on the photoacoustic and/or ultrasound data.

For example, as described herein, some implementations relate to a biomedical instrument that can be used to monitor comprehensive patient conditions during labor (e.g., fetal O₂Satₛₛ, heart rate, cervical dilation, and/or uterine contractions), which has not previously been achieved by any other single-modal or multi-modal approach. For example, although EFM can be used to monitor a fetal heartbeat and uterine contractions using Doppler and tocodynometer sensors, EFM cannot monitor fetal brain oxyhemoglobin saturation and cervical dilation. Furthermore, although clinical ultrasound imaging can measure various fetal heart functions and cervical dilation, ultrasound imaging cannot provide molecular contrast to quantify O₂Satₛₛ. In addition, NIRS may measure fetal O₂Satₛₛ, but the NIRS modality cannot perform anatomical imaging of cervical dilation and fetal heart sensing. The photoacoustic monitoring provided by the device described herein combines features attributed to both light and sound, which includes high contrast and versatility in sensing biological targets, as well as excellent spatial resolution not compromised by light scattering, and a relatively low implementation cost. Additionally, multispectral measurements may be used to enable quantitative sensing of tissue chromophores based on their optical absorption spectrum.

In some implementations, as shown by reference number 530 in FIG. 5C, a system for noninvasive biological function monitoring may comprise a hardware platform, which may include a soft and compact endovaginal light guide that delivers a safe amount of pulsed light energy at multiple NIR wavelengths. The excitation laser source for the endovaginal light guide can be a compact pulsed light-emitting diode (LED) or pulsed laser diode (PLD), which is much smaller, safer, and requires less power than a conventional laser. The use of a low-power PLD or LED, separated from an acoustic sensor, may result in the endovaginal component being as compact as possible to be a wearable form factor, while avoiding any electronic or mechanical parts inside the patient's body. As further shown in FIG. 5C, the hardware platform may include a transabdominal scanner with a 2-degree-of-freedom (DOF) that performs ultrasound and photoacoustic imaging over wide FOV, covering a cervix, a fetal head, and a fetal heart. The robotized configuration of the transabdominal scanner may provide accurate volumetric scanning and more versatile adjustment in dynamic laboring scenarios. The hardware platform may also allow manual control whenever and wherever a clinician needs to scan a specific location (e.g., of the fetal head or heart) in real-time. In addition, the hardware platform of the device may include a hybrid power supply, which may be wired and charging when patients are in bed and near an available (e.g., wall-plugged) power source, and detached for freedom of movement with a battery life greater than one hour when the power source is unavailable (e.g., allowing the pregnant woman to move freely during labor). An ultrasound receiver module with an efficient architecture and algorithms may be used with state-of-art electronic chip integration technologies, and a uterine contraction sensor may be used for temporal correlation of fetal O₂Satₛₛ and heart rate with uterine contractions, thereby enabling a better understanding of fetal O₂Satₛₛ, heart rate, and/or other metrics with respect to fetal brain health.

As further shown in FIG. 5C, and by reference number 532, the device described herein may include one or more real-time software components, which may enable continuous placement and tracking of regions-of-interest (ROIs) at fetal head and cervix (e.g., as a “virtual helmet”) and a fetal heart (e.g., as a “virtual catheter”) at any arbitrary scanning direction of the transabdominal scanner. The continuous placement and tracking of ROIs may provide essential FOV and fast scanning rate at the same time. In addition, the software components may enable autonomous and continuous processing of virtual helmet and virtual catheter ROIs by a deep learning (DL) agent, which may result in robust measurement of fetal O₂Satₛₛ, heart rate, and cervical dilation. The endovaginal light guide may include a structural marker to define the relative angle and position of the virtual helmet. Anatomical features of the fetal heart may serve as a structural marker for the virtual catheter. In this way, the automation in labor progress monitoring may simplify and ease the clinical protocol and burden. For example, frequent cervical palpation, which has produced patient discomfort and increased in-person clinician workload, may be rendered unnecessary. Furthermore, an intrusive fetal scalp electrode, a current commonly used method to confirm an abnormal fetal heart rate (FHR), will not be needed to confirm FHR abnormalities. In addition, clinically relevant spatial resolution by the small wearable form factor can be secured with a set of imaging algorithms such as 2-DOF (e.g., rotational and translational) synthetic aperture focusing (SAF) techniques based on an analytical solution may be used to define spatial resolution and grating lobe positions. For example, as shown by reference number 534-1, the software components may be used to perform preclinical validation based on an estimated oxygen saturation as a function of measured oxygen saturation. Additionally, or alternatively, as shown by reference number 534-2, the software components may be used for autonomous extraction of target plane data from three-dimensional (3D) data, and/or to image a target monitoring window, as shown by reference number 534-3. In this way, the hardware and software platform can derive various metrics related to fetal health (or other patient health), as shown by reference number 536. For example, as shown by reference numbers 538-1 and 538-2, the metrics may include antepartum fetal evaluation metrics, such as fetal biometrics and/or a fetal heart rate. Additionally, or alternatively, as shown by reference numbers 538-2, 538-3, and 538-4, the metrics may include intrapartum fetal and/or labor monitoring metrics, such as a fetal heart rate, a fetal O₂Satₛₛ, and/or a cervical dilation, among other examples.

In addition, as described herein, the photoacoustic and/or ultrasound monitoring device is a wearable device that has a potential broader impact on fetal health across gestational age. For example, in some implementations, the photoacoustic and/or ultrasound monitoring device may be used as a wearable device to remotely identify fetal growth restriction (FGR) remotely. Moreover, the photoacoustic and/or ultrasound monitoring device may be used as a co-robotic ultrasound wearable device that allows steady ultrasound imaging with reduced motion artifacts, enabling slow flow imaging algorithms with high degree of accuracy and fidelity.

Accordingly, in some implementations, the photoacoustic and/or ultrasound monitoring device described herein may provide a rigorous linkage between cerebrovascular physiology and brain injury in practical scenarios based on HI brain distress at different severities, durations, and periodicities. Some implementations described herein may couple various HI distresses, reflected at O₂Satₛₛ, with an extent of neuronal death in follow-up histopathological studies. The database may provide a reference source to notify clinicians for necessary actions in time, and clinical interfaces may combine such multi-dimensional factors to provide intuitive and effective alarms to prevent fetal brain injury based on the relationship of the time course of O₂Satₛₛ with the probability of neuronal loss. For example, as described herein, a wearable photoacoustic and ultrasound monitoring system may include a monitoring device and a control device that can be used to continuously report fetal sagittal sinus venous HbO₂ saturation, fetal heart function, and/or cervical dilation during labor, where the continuous monitoring may be used to track the intensity and duration of fetal brain deoxygenation resulting from decreases in fetal arterial desaturation and/or cerebral blood flow. In contrast, cardiac decelerations reported by EFM are sensitive to transient reductions in fetal arterial partial pressure of oxygen (pO₂) (e.g., the amount of oxygen gas dissolved in blood) during uterine contractions, but are less sensitive to steady-state reductions in fetal pO₂ or reductions in cerebral blood flow caused by arterial hypotension or skull compression. Thus, the photoacoustic and ultrasound measurement of fetal cerebral venous O₂ saturation enabled by the wearable photoacoustic and ultrasound monitoring device may be superior to EFM in predicting the likelihood of neuronal dysfunction and eventual necrosis of biological tissue. Furthermore, the system may be used to monitor a pregnant woman during labor, identifying at-risk fetal brain, heart function, the progress of cervical dilation, and machine learning-driven visible/audible feedback to the clinician whenever fetal distress and/or failure for labor progress is identified. The wearable photoacoustic and ultrasound monitoring device may also decrease a clinician workload and physical/mental discomfort of the pregnant woman due to frequent palpation to evaluate cervical dilation.

For example, referring to FIG. 5D, reference numbers 540 and 542 illustrate a wearable clinical device that can be used to monitor fetal sagittal sinus O₂Satₛₛ, fetal heart function, and maternal cervical dilation for autonomous labor monitoring and accurate clinical decision making for follow-up interventions and treatments. For example, as shown by reference number 540, the wearable clinical device may include an ultrasound scanner and light source, which may include or may be coupled to one or more batteries. In some implementations, the ultrasound scanner and light source may be worn externally (e.g., on an abdomen of a pregnant woman). Furthermore, as shown by reference number 542, the device may include an endovaginal light guide that is inserted internally (e.g., within a cervical canal of a pregnant woman) to enable monitoring of fetal brain health, fetal heart health, and/or cervical dilation. For example, as shown by reference number 544, the endovaginal light guide may include a reusable light guide. As further shown by reference number 546, the endovaginal light guide may include a disposable balloon cover that may allow light to travel through the cover and into the tissue to be monitored. Furthermore, as shown by reference number 548, the endovaginal light guide may include a rubberized ring, such as a position and angle indicator ring, for controlling a position and angle of the endovaginal light guide. As further shown in FIG. 5D, the ultrasound scanner and light source may be a non-disposable (e.g., reusable) component. Furthermore, the endovaginal light guide may include one or more non-disposable (e.g., reusable) components, such as the reusable light guide, and one or more disposable (e.g., one-time use) components, such as the disposable balloon cover and the rubberized ring. As described herein, the light source may emit light energy that the endovaginal light guide guides toward the tissue (e.g., via an optical fiber bundle) as pulsed light energy at multiple NIR wavelengths. Furthermore, the endovaginal light guide may include a housing (e.g., for the optical fiber bundle) made from a transparent or translucent material that enables the light energy to be emitted through the housing toward the tissue. Accordingly, the pulsed light that is generated by the light source worn externally and carried into the birth canal via the endovaginal light guide may be absorbed by a chromophore, such as hemoglobin, thereby generating an acoustic pressure that corresponds with the light absorbance of the target region. The generated acoustic pressure propagates through biological tissue and is then measured by the external ultrasound transducer (or ultrasound scanner). In this way, the measured acoustic pressure can enable spectroscopic quantification of HbO₂, which may be used to monitor fetal brain oxygenation as a direct and early indicator of brain injury due to HIE and stroke.

In some implementations, as described herein, the photoacoustic and/or ultrasound monitoring device may include a 2-DOF transabdominal scanner integrated with the endovaginal light source. The kinematic module may support 2 DOF in translation and rotation, which may provide 50-mm and 30° scanning by a phased array. In some implementations, the photoacoustic and/or ultrasound monitoring device may be associated with minimal requirements for linear and rotational velocity at 15 mm/sec and 15°/sec, leading to theoretical full scanning time at 5 sec. A mechatronic design goal is to have a low-profile system volume to fully accommodate patient freedom of movement. A current target system volume is 80×50×30 mm³ (e.g., width, depth, height). Note that moving parts will not have any physical contact with a patient abdomen and instead will employ an acoustically semi-transparent frame made by polymethylpentene filled with an acoustic couplant. The kinematic module may be integrated to a customized ultrasound array connected to a multi-channel data acquisition system.

In some implementations, as described herein, the endovaginal light guide may be used to deliver NIR light from the light source that is worn externally into the birth canal. In addition, the endovaginal light guide may define the virtual helmet for fetal brain and/or cervical monitoring. For example, the disposable balloon cover for the endovaginal light guide may contain the pessary-type rubberized ring, shown by reference number 548, to indicate the position and angle of the endovaginal light guide. The ring may provide a unique structural pattern in an anatomical ultrasound image. Furthermore, the endovaginal light guide may support 2-DOF motorized maneuvering to adjust the position and/or angle and consistently target the fetal SSS region, which can be localized by 3D ultrasound image and photoacoustic intensity.

In some implementations, an imaging sequence of the wearable photoacoustic and ultrasound monitoring device may follow an intermittent auscultation (IA) protocol established by the American College of Nurse-Midwives (ACNM). For example, in the IA protocol, low-risk laboring patients may have 30 minute intervals for fetal evaluation in a first stage of labor (e.g., before full cervical dilation), and the monitoring frequency can be increased to every 15 minutes in a second stage of labor (e.g., when fully dilated at an approximately 10 centimeter diameter). On the other hand, high-risk patients may have more frequent monitoring every 15 minutes and every 5 minutes for the first stage and the second stage of labor, respectively. In some implementations, the control device may display an interface that allows a clinician to categorize a laboring patient as experiencing low-risk or high-risk labor, and the monitoring interval may be adjusted according to the cervical dilation measured in the 3D ultrasound image obtained by the ultrasound scanner. In each monitoring interval, each ROI may be associated with a different temporal scanning requirement. For example, in some implementations, a fetal heart can have 110-160 beats per minute, whereby successful heart rate quantification may be enabled by a >2× temporal scanning rate (e.g., 220-320 beats per minute, or 3.7-5.3 scans per second). However, cervical dilation is a slow process, whereby one volumetric ultrasound image per 5-30 minutes may marginally track the change in cervical dilation. Photoacoustic sensing of fetal SSS may necessitate multiple pulsed light illumination for spectral scanning and frame averaging, whereby each scanning sequence during a monitoring bin (e.g., 5, 10, or 15 min) may include a full ultrasound scan for approximately 20 seconds, fetal heart M-mode imaging for approximately 2 seconds (e.g., about 4 heartbeat cycles), and fetal SSS monitoring for approximately 4 seconds (e.g., a 1 millisecond pulse interval (1 kHz pulse repetition frequency)×2 wavelengths×2000 frame averaging). In addition, the control device and/or the monitoring device may support intermittent partial scan sessions that may measure one or more fetus-related metrics (e.g., O₂Satₛₛ and/or heart rate) that may be triggered by uterine contraction peaks (e.g., because the presence of brain HI is highest right after the contraction). Furthermore, a mechanical scanning sequence between scan sessions may be optimized to minimize latency. For example, FIG. 5E illustrates an example of the monitoring sequence, where reference number 550 corresponds to a regular full scan session that delineates all metrics being monitored and the anatomy in an entire FOV. Furthermore, reference number 552 corresponds to intermittent partial scan sessions that may be used to measure fetus-related metrics when uterine contraction peaks, as shown by reference number 554. Furthermore, in each regular scan session, reference number 556 corresponds to an adjustment period for the ROI (e.g., to adjust the endovaginal light guide targeting the SSS).

In some implementations, the wearable photoacoustic and ultrasound monitoring device may have a kinematic design that can provide 2-DOF in volumetric scanning using a clinical phased array. For example, in some implementations, the 2-DOF motion may be achieved with a parallel mechanism, where the externally worn ultrasound scanner includes an ultrasound array attached to a link that spans two parallel linear stages. The link may be connected to the first linear stage with a revolving joint and to the second linear stage with a pin slot joint. Accordingly, a common motion of the two linear stages may cause the array to translate and a differential motion may cause the transducer to rotate. The kinematics may be similar to a prismatic joint followed by a rotation joint, where the two linear stage positions are denoted a q₁ and q₂, , respectively, and the minimum distance between the pins on the two linear stages is denoted dₚ, resulting in forward kinematics equated as:

$${T}_{0}^{US} = [ \begin{matrix}cos ( {θ}_{1} ) & - sin ( {θ}_{1} ) & 0 & {d}_{1} \\ sin ( {θ}_{1} ) & cos ( {θ}_{1} ) & 0 & 0 \\ 0 & 0 & 1 & 0 \\ 0 & 0 & 0 & 1\end{matrix} ] {T}_{1}^{US}$$

where d₁=q₁, θ₁=atan2(dₚ, q₂−q₁), and T₁^{US} is a constant dependent on the mounting position of the transducer. Furthermore, the external component may include a wedge-shaped elastic gel pad to maintain acoustic coupling. Accordingly, the kinematic design allows anatomical features of the target to be well-delineated by multi-angle synthesis.

FIG. 5F illustrates an example of effective points of sensing (PoS) for a FOV containing a fetal brain, a fetal heart, and a cervix. In particular, FIG. 5F depicts results of a feasibility case test of the PoS, which indicates that a single 2-DOF transabdominal scanner can cover a wide enough FOV for the virtual helmet for a fetal brain/cervix and the virtual catheter for a fetal heart. For example, reference number 560 corresponds to MRI data of a pregnant woman at 34-week's gestation that contains an entire anatomy from a maternal abdominal surface to the entire fetal body. As shown by reference number 560, the MRI data shows two target ROIs, including an interface point between the cervix and the fetal head and the center of the fetal heart. After refining the MRI data in a meshed space, each PoS grid was tested to assess whether the given 2-DOF of the wearable photoacoustic and ultrasound monitoring device can cover the target ROIs. For example, as shown by reference number 562, a meshed abdomen surface and scanning FOV at each grid PoS indicates a PoS for the fetal head and a PoS for the fetal heart. Accordingly, reference number 564 depicts an overlapped PoS map to cover both target ROIs (e.g., for the fetal head and the fetal heart), which indicates that a wide lower abdominal region of the pregnant mother is an optimal position to place the 2-DOF transabdominal scanner that the pregnant mother wears externally.

FIG. 5G illustrates an example of deep learning-based autonomous target ROI tracking and measurement software that may be supported by the control device. For example, the control device may acquire a complete abdominal volume from the wearable photoacoustic and ultrasound monitoring device, and a deep learning (DL) agent running on the control device may automatically detect one or more trained target ROIs to localize the cervical-fetal head interface (e.g., by tracking the pessary-type rubberized indicator ring shown in FIG. 5D and described in more detail above). Additionally, or alternatively, the DL agent may localize the center of the fetal heart in the abdominal volume. For example, in some implementations, a 3D landmark positioning network can be used to identify a coarse ROI for the brain/cervical region and a coarse ROI for the heart region. As shown in FIG. 5G, and by reference number 570, a deep Q-network (DQN) agent may model a process of iteratively searching for a target 2D view from a 3D sub-volume of the acquired data (e.g., corresponding to the fetal head/cervix ROI). In some implementations, as shown, the DQN agent may include a slice generator, a policy network, and a selection function that may obtain one or more key parameters, such as a cervical opening via a U-Net-based segmentation algorithm. Furthermore, in some implementations, an identified ROI location can also be used for guiding the imaging direction to acquire M-mode images in the heart region for heart-rate monitoring.

Furthermore, for searching the target 2D view from the 3D sub-volume, the DQN agent may produce, given a random initial pose of the starting plane, a rapid discrete action that moves the plane one step closer to the target based on prior knowledge of the 2-DOF kinematic. For example, given any initial state θᵢ, the 2D planar resampler may be transformed, interpolating the 3D volume and outputting a 2D cross-section image of the volume. The policy network may take the resampled 2D image as an input and output one or more Q-values corresponding to taking different actions. Then, the action with a largest Q-value will be taken to transition to the next state θᵢ₊₁. For example, reference number 572 depicts an example where a trained DL agent has extracted a target plane from a large test 3D ultrasound data. As further shown by reference number 574, mean translational and rotational errors are significantly lower than a control case without the DQN agent. In some implementations, the DQN-based searching algorithm can be trained and evaluated on a 3D head volume ultrasound dataset. Furthermore, in some implementations, the DL agent may integrate one or more optimized photoacoustic/ultrasound spatial-spectral imaging algorithms.

As indicated above, FIGS. 5A-5G are provided as examples. Other examples may differ from what is described with regard to FIGS. 5A-5G.

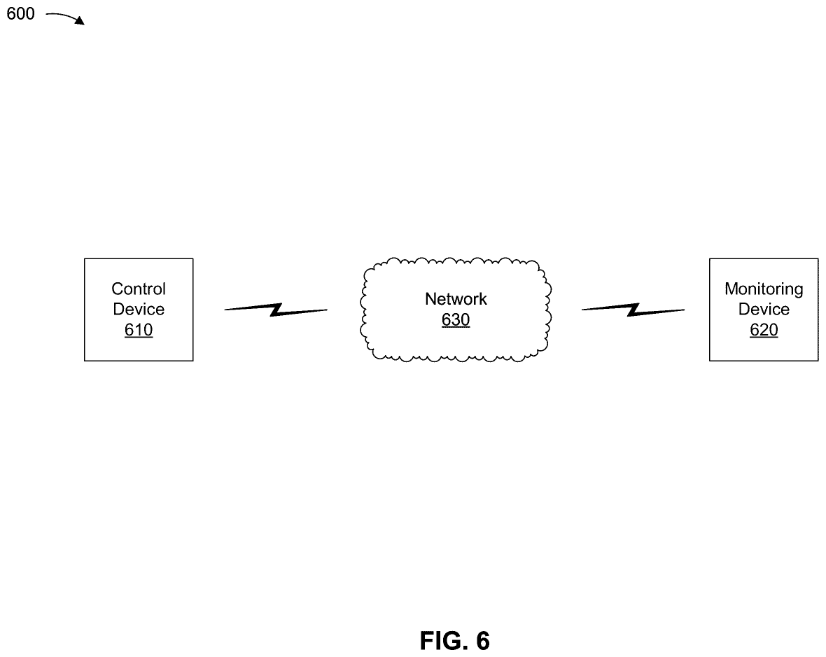

FIG. 6 is a diagram of an example environment 600 in which systems and/or methods described herein may be implemented. As shown in FIG. 6, environment 600 may include a control device 610, a monitoring device 620, and a network 630. Devices of environment 600 may interconnect via wired connections, wireless connections, or a combination of wired and wireless connections.

Control device 610 includes one or more devices capable of receiving, generating, storing, processing, and/or providing information associated with monitoring device 620 and/or providing imaging of tissue. For example, control device 610 may include a communication and/or computing device, such as a computer (e.g., a laptop computer, a tablet computer, a handheld computer, a desktop computer), a mobile phone (e.g., a smart phone), a wearable device (e.g., a smart wristwatch, a pair of smart eyeglasses, a heads-up display device, a virtual reality device, or a visual augmentation device), or a similar type of device. In some implementations, control device 610 includes one or more devices to control monitoring device 620, such as a control console, a telemanipulator, an end-effector, and/or a remote surgery console. In some implementations, control device 610 may include a user interface (e.g., a display device) for providing a visualization of imaging data, an image processing device for processing the imaging data to generate the visualization, and/or the like. In some implementations, control device 610 corresponds to control device 110 shown in FIG. 1.

Monitoring device 620 includes one or more devices capable of receiving, generating, storing, processing, and/or providing information associated with imaging and/or monitoring a patient. For example, monitoring device 620 may include an ME component, an ultrasound sensing component, an ultrasound transmission component, a light source, an endovaginal light guide, and/or the like. Although some implementations described herein are described in terms of an integrated monitoring device to monitor a patient, some implementations described herein may be used to obtain imaging data from a dedicated imaging device based on one or more operations to enable capturing of the imaging data. In some implementations, monitoring device 620 corresponds to monitoring device 120 shown in FIG. 1.

Network 630 includes one or more wired and/or wireless networks. For example, network 630 may include a cellular network (e.g., a long-term evolution (LTE) network, a code division multiple access (CDMA) network, a 3G network, a 4G network, a 5G network, a 6G network, another type of next generation network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, a cloud computing network, or the like, and/or a combination of these or other types of networks.

The number and arrangement of devices and networks shown in FIG. 6 are provided as an example. In practice, there may be additional devices and/or networks, fewer devices and/or networks, different devices and/or networks, or differently arranged devices and/or networks than those shown in FIG. 6. Furthermore, two or more devices shown in FIG. 6 may be implemented within a single device, or a single device shown in FIG. 6 may be implemented as multiple, distributed devices. Additionally, or alternatively, a set of devices (e.g., one or more devices) of environment 600 may perform one or more functions described as being performed by another set of devices of environment 600.

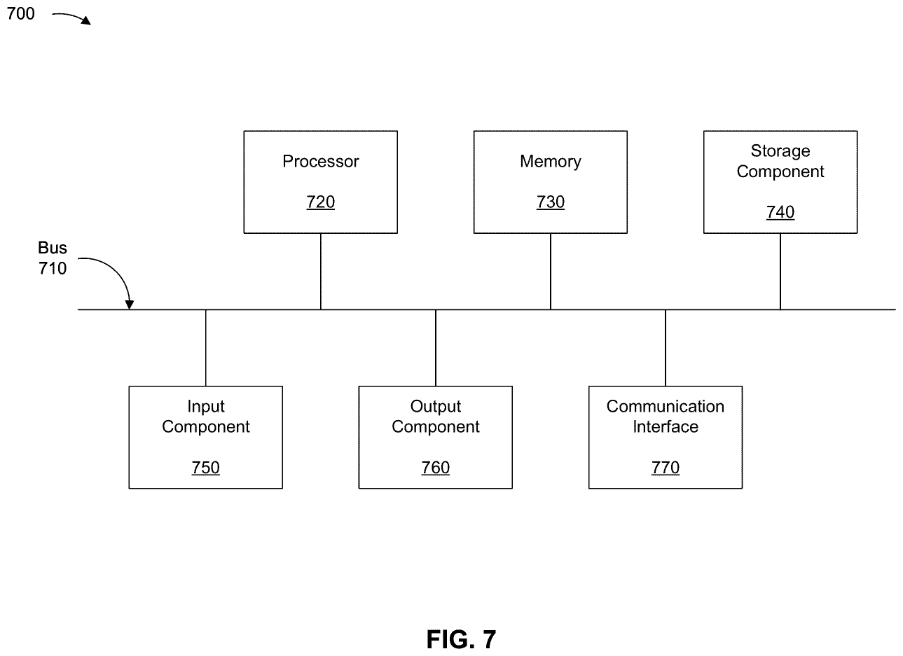

FIG. 7 is a diagram of example components of a device 700. Device 700 may correspond to control device 610 and/or monitoring device 620. In some implementations, control device 610 and/or monitoring device 620 may include one or more devices 700 and/or one or more components of device 700. As shown in FIG. 7, device 700 may include a bus 710, a processor 720, a memory 730, a storage component 740, an input component 750, an output component 760, and/or a communication interface 770.

Bus 710 includes a component that permits communication among multiple components of device 700. Processor 720 is implemented in hardware, firmware, and/or a combination of hardware and software. Processor 720 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 720 includes one or more processors capable of being programmed to perform a function. Memory 730 includes a random access memory (RAM), a read only memory (ROM), and/or another type of dynamic or static storage device (e.g., a flash memory, a magnetic memory, and/or an optical memory) that stores information and/or instructions for use by processor 720.

Storage component 740 stores information and/or software related to the operation and use of device 700. For example, storage component 740 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

Input component 750 includes a component that permits device 700 to receive information, such as via user input (e.g., a touch screen display, a keyboard, a keypad, a mouse, a button, a switch, and/or a microphone). Additionally, or alternatively, input component 750 may include a sensor for sensing information (e.g., a global positioning system (GPS) component, an accelerometer, a gyroscope, and/or an actuator). Output component 760 includes a component that provides output information from device 700 (e.g., a display, a speaker, and/or one or more light-emitting diodes (LEDs)).

Communication interface 770 includes a transceiver-like component (e.g., a transceiver and/or a separate receiver and transmitter) that enables device 700 to communicate with other devices, such as via a wired connection, a wireless connection, or a combination of wired and wireless connections. Communication interface 770 may permit device 700 to receive information from another device and/or provide information to another device. For example, communication interface 770 may include an Ethernet interface, an optical interface, a coaxial interface, an infrared interface, a radio frequency (RF) interface, a universal serial bus (USB) interface, a Wi-Fi interface, a cellular network interface, or the like.

Device 700 may perform one or more processes described herein. Device 700 may perform these processes based on processor 720 executing software instructions stored by a computer-readable medium, such as memory 730 and/or storage component 740. A computer-readable medium is defined herein as a non-transitory memory device. A memory device includes memory space within a single physical storage device or memory space spread across multiple physical storage devices.

Software instructions may be read into memory 730 and/or storage component 740 from another computer-readable medium or from another device via communication interface 770. When executed, software instructions stored in memory 730 and/or storage component 740 may cause processor 720 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of components shown in FIG. 7 are provided as an example. In practice, device 700 may include additional components, fewer components, different components, or differently arranged components than those shown in FIG. 7. Additionally, or alternatively, a set of components (e.g., one or more components) of device 700 may perform one or more functions described as being performed by another set of components of device 700.

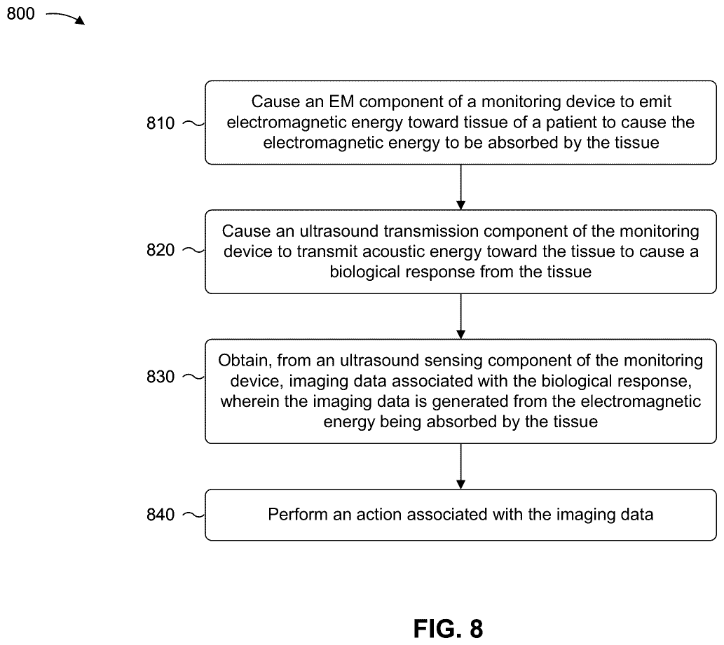

FIG. 8 is a flow chart of an example process 800 associated with biological function monitoring. In some implementations, one or more process blocks of FIG. 8 may be performed by a control device (e.g., control device 810). In some implementations, one or more process blocks of FIG. 8 may be performed by another device or a group of devices separate from or including the monitoring device, such as a monitoring device (e.g., monitoring device 820) and/or the like.

As shown in FIG. 8, process 800 may include causing an EM component of a monitoring device to emit energy toward tissue of a patient to cause the energy to be absorbed by the tissue (block 810). For example, the control device (e.g., using processor 720, memory 730, storage component 740, output component 760, communication interface 770, and/or the like) may cause an EM component of a monitoring device to emit energy toward tissue of a patient to cause the energy to be absorbed by the tissue, as described above.

As further shown in FIG. 8, process 800 may include causing an ultrasound transmission component to transmit acoustic energy toward the tissue to cause a biological response from the tissue (block 820). For example, the control device (e.g., using processor 720, memory 730, storage component 740, output component 760, communication interface 770, and/or the like) may cause an ultrasound transmission component to transmit acoustic energy toward the tissue to cause a biological response from the tissue, as described above.

As further shown in FIG. 8, process 800 may include obtaining, from an ultrasound sensing component, imaging data associated with the biological response, wherein the imaging data is generated from the energy being absorbed by the tissue (block 830). For example, the control device (e.g., using processor 720, memory 730, storage component 740, input component 750, communication interface 770, and/or the like) may obtain, from an ultrasound sensing component, imaging data associated with the biological response, as described above. In some implementations, the imaging data is generated from the energy being absorbed by the tissue.

As further shown in FIG. 8, process 800 may include performing an action associated with the imaging data (block 840). For example, the control device (e.g., using processor 720, memory 730, storage component 740, output component 760, communication interface 770, and/or the like) may perform an action associated with the imaging data, as described above.

Process 800 may include additional implementations, such as any single implementation or any combination of implementations described below and/or in connection with one or more other processes described elsewhere herein.

In some implementations, the control device, when causing the EM component to emit light pulses, may cause the EM component to emit light pulses with a threshold frequency to enable detection of the biological response in an image generated from the imaging data. In some implementations, the control device, when causing the ultrasound transmission component to transmit acoustic energy, may cause the ultrasound transmission component to transmit the acoustic energy at a neuromodulation frequency. In some implementations, the control device may cause the ultrasound sensing component to generate the imaging data associated with the biological response.

In some implementations, the imaging data corresponds to data for an image stream, and the control device may cause the ultrasound sensing component to generate the imaging data to have a threshold spatial resolution of the image stream and/or to have a threshold frame rate of the image stream. In some implementations, the control device may measure a parameter associated with the biological response based on the imaging data and determine a status of the tissue based on the parameter. In some implementations, the action is performed based on the status of the tissue.

In some implementations, the control device, when performing the action, may indicate the status of the tissue via a user interface communicatively coupled with the device. In some implementations, the control device, when performing the action, may cause an image generated from the imaging data to be displayed via a user interface communicatively coupled with the device. In some implementations, the optical or microwave component, the ultrasound transmission component, and the ultrasound sensing component are components of a photoacoustic device.

In some implementations, the control device may obtain, from an electroencephalography (EEG) component, electrical activity data associated with the biological response, and determine timing associated with the biological response based on the electrical activity data. In some implementations, the electrical activity data is obtained from the EEG component based on the timing associated with the biological response.

In some implementations, the control device may measure a parameter associated with the biological response based on the imaging data and the electrical activity data, and determine a status of the tissue based on the parameter. In some implementations, the action is performed based on the status of the tissue. In some implementations, the control device may use a machine learning model to identify a value associated with the parameter. In some implementations, the machine learning model is trained based on historical data associated with measuring the parameter from other imaging data and/or other electrical activity data associated with one or more other corresponding biological responses incited in one or more other corresponding tissues of one or more other patients.

Although FIG. 8 shows example blocks of process 800, in some implementations process 800 may include additional blocks, fewer blocks, different blocks, or differently arranged blocks than those depicted in FIG. 8. Additionally, or alternatively, two or more of the blocks of process 800 may be performed in parallel.

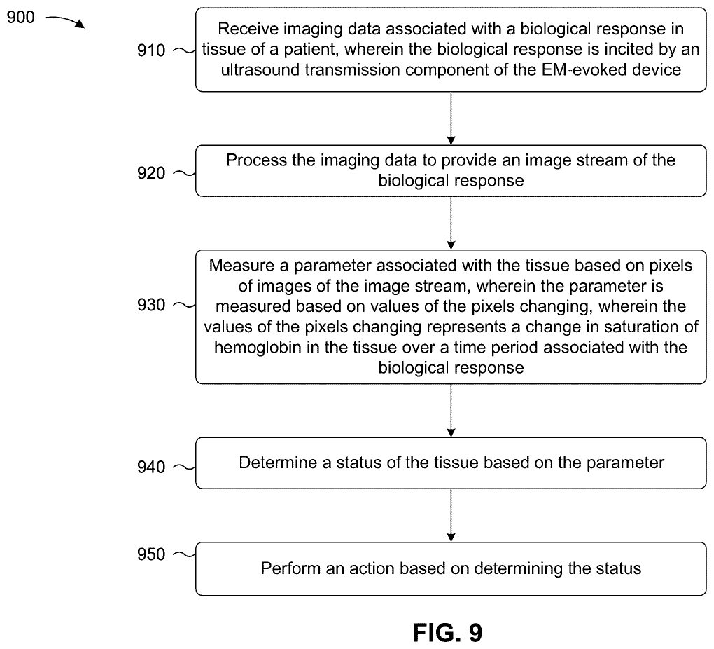

FIG. 9 is a flow chart of an example process 900 associated with photoacoustic or thermoacoustic monitoring. In some implementations, one or more process blocks of FIG. 9 may be performed by a control device (e.g., control device 710). In some implementations, one or more process blocks of FIG. 9 may be performed by another device or a group of devices separate from or including the monitoring device, such as a monitoring device (e.g., monitoring device 720) and/or the like.

As shown in FIG. 9, process 900 may include receiving, from an EM-evoked device, imaging data associated with a biological response in tissue of a patient, wherein the biological response is incited by an ultrasound transmission component of the photoacoustic device (block 910). For example, the control device (e.g., using processor 720, memory 730, storage component 740, input component 750, communication interface 770, and/or the like) may receive, from a photoacoustic device, imaging data associated with a biological response in tissue of a patient, as described above. In some implementations, the biological response is incited by an ultrasound transmission component of the photoacoustic device.

As further shown in FIG. 9, process 900 may include processing the imaging data to provide an image stream of the biological response (block 920). For example, the control device (e.g., using processor 720, memory 730, storage component 740, input component 750, output component 760, communication interface 770, and/or the like) may process the imaging data to provide an image stream of the biological response, as described above.

As further shown in FIG. 9, process 900 may include measuring a parameter associated with the tissue based on pixels of images of the image stream, wherein the parameter is measured based on values of the pixels changing, wherein the values of the pixels changing represents a change in saturation of hemoglobin in the tissue over a time period associated with the biological response (block 930). For example, the control device (e.g., using processor 720, memory 730, storage component 740, input component 750, output component 760, communication interface 770, and/or the like) may measure a parameter associated with the tissue based on pixels of images of the image stream, as described above. In some implementations, the parameter is measured based on values of the pixels changing. In some implementations, the values of the pixels changing represents a change in saturation of hemoglobin in the tissue over a time period associated with the biological response.

As further shown in FIG. 9, process 900 may include determining a status of the tissue based on the parameter (block 940). For example, the control device (e.g., using processor 720, memory 730, storage component 740, input component 750, output component 760, communication interface 770, and/or the like) may determine a status of the tissue based on the parameter, as described above.

As further shown in FIG. 9, process 900 may include performing an action based on determining the status (block 950). For example, the control device (e.g., using processor 720, memory 730, storage component 740, input component 750, output component 760, communication interface 770, and/or the like) may perform an action based on determining the status, as described above.

Process 900 may include additional implementations, such as any single implementation or any combination of implementations described below and/or in connection with one or more other processes described elsewhere herein.

In some implementations, the values of the pixels changing further represents at least one of a change in cytochrome aa3 oxidized state in the tissue over a time period associated with the biological response, or a change in lipid content of the tissue over a time period associated with the biological response.

In some implementations, the control device, when measuring the parameter, may use a machine learning model to identify a value associated with the parameter, wherein the machine learning model is trained based on historical data associated with measuring the parameter from other imaging data associated with one or more other corresponding biological responses incited in one or more other corresponding tissues of one or more other patients.

In some implementations, the control device, when determining the status of the tissue, may determine that a value associated with the parameter satisfies a threshold, and determine that the status is hypoxic based on the value associated with the parameter satisfying the threshold. In some implementations, the action is performed based on determining that the status is hypoxic. In some implementations, the control device, when performing the action, may issue, via a user interface, an alert associated with the patient based on determining that the status is hypoxic.

In some implementations, the control device, when performing the action, may indicate the status of the tissue via a user interface communicatively coupled with the device. In some implementations, a value of the parameter indicates a probability that the patient is experiencing a stroke. In some implementations, the tissue may be brain tissue. In some implementations, the patient is a fetus. In some implementations, the fetus is at least partially in a uterus of another patient.

In some implementations, a device with the proposed configurations can be used for other clinical applications with endoscopic or endorectal configurations. Such a configuration may support monitoring brain stem hypoxia; detecting brain death in critically ill patients; and evaluating patients for intra-abdominal bleeding.

Although FIG. 9 shows example blocks of process 900, in some implementations, process 900 may include additional blocks, fewer blocks, different blocks, or differently arranged blocks than those depicted in FIG. 9. Additionally, or alternatively, two or more of the blocks of process 900 may be performed in parallel.

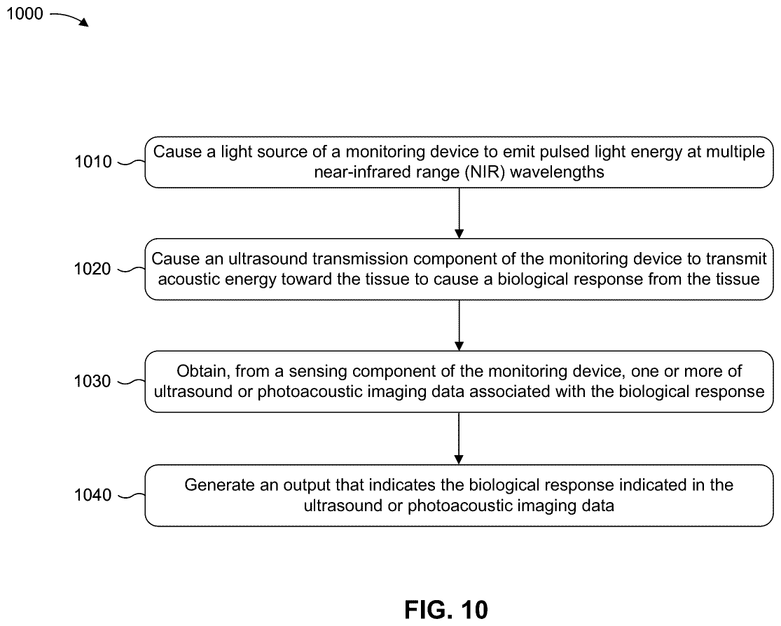

FIG. 10 is a flowchart of an example process 1000 associated with a wearable ultrasound and photoacoustic device for fetal and/or labor monitoring. In some implementations, one or more process blocks of FIG. 10 are performed by a control device (e.g., control device 610). In some implementations, one or more process blocks of FIG. 10 are performed by another device or a group of devices separate from or including the control device, such as monitoring device 620. Additionally, or alternatively, one or more process blocks of FIG. 10 may be performed by one or more components of device 700, such as processor 720, memory 730, storage component 740, output component 760, and/or communication interface 770.

As shown in FIG. 10, process 1000 may include causing a light source of a monitoring device to emit pulsed light energy at multiple NIR wavelengths, wherein the light source is coupled to a light-guiding component that guides the light energy toward tissue of a patient to cause the light energy to be absorbed by the tissue (block 1010). For example, the control device may cause a light source of a monitoring device to emit pulsed light energy at multiple NIR wavelengths, wherein the light source is coupled to a light-guiding component that guides the light energy toward tissue of a patient to cause the light energy to be absorbed by the tissue, as described above. In some implementations, the light source is coupled to a light-guiding component that guides the light energy toward tissue of a patient to cause the light energy to be absorbed by the tissue.

As further shown in FIG. 10, process 1000 may include causing an ultrasound transmission component of the monitoring device to transmit acoustic energy toward the tissue to cause a biological response from the tissue (block 1020). For example, the control device may cause an ultrasound transmission component of the monitoring device to transmit acoustic energy toward the tissue to cause a biological response from the tissue, as described above.

As further shown in FIG. 10, process 1000 may include obtaining, from a sensing component of the monitoring device, one or more of ultrasound or photoacoustic imaging data associated with the biological response, wherein the ultrasound or photoacoustic imaging data is generated from the light energy being absorbed by the tissue (block 1030). For example, the control device may obtain, from a sensing component of the monitoring device, one or more of ultrasound or photoacoustic imaging data associated with the biological response, wherein the ultrasound or photoacoustic imaging data is generated from the light energy being absorbed by the tissue, as described above. In some implementations, the ultrasound or photoacoustic imaging data is generated from the light energy being absorbed by the tissue.

As further shown in FIG. 10, process 1000 may include generating an output that indicates the biological response indicated in the ultrasound or photoacoustic imaging data (block 1040). For example, the control device may generate an output that indicates the biological response indicated in the ultrasound or photoacoustic imaging data, as described above.

Process 1000 may include additional implementations, such as any single implementation or any combination of implementations described below and/or in connection with one or more other processes described elsewhere herein.

In a first implementation, the light source includes one or more pulsed LEDs or PLDs.

In a second implementation, alone or in combination with the first implementation, the ultrasound transmission component, the sensing component, and the light source are included in a housing that is wearable on an abdomen or other external body region relative to the tissue associated with the ultrasound or photoacoustic imaging data.

In a third implementation, alone or in combination with one or more of the first and second implementations, the monitoring device includes one or more batteries, included in or coupled to a housing that includes the ultrasound transmission component, the sensing component, and the light source, to receive and store electrical energy.

In a fourth implementation, alone or in combination with one or more of the first through third implementations, the ultrasound or photoacoustic imaging data indicates a position and an angle of a rubberized ring of the light-guiding component.

Although FIG. 10 shows example blocks of process 1000, in some implementations, process 1000 includes additional blocks, fewer blocks, different blocks, or differently arranged blocks than those depicted in FIG. 10. Additionally, or alternatively, two or more of the blocks of process 1000 may be performed in parallel.

The foregoing disclosure provides illustration and description, but is not intended to be exhaustive or to limit the implementations to the precise form disclosed. Modifications and variations may be made in light of the above disclosure or may be acquired from practice of the implementations.

As used herein, the term “component” is intended to be broadly construed as hardware, firmware, and/or a combination of hardware and software.

Some implementations are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, or the like, depending on the context.

Certain user interfaces have been described herein and/or shown in the figures. A user interface may include a graphical user interface, a non-graphical user interface, a text-based user interface, or the like. A user interface may provide information for display. In some implementations, a user may interact with the information, such as by providing input via an input component of a device that provides the user interface for display. In some implementations, a user interface may be configurable by a device and/or a user (e.g., a user may change the size of the user interface, information provided via the user interface, a position of information provided via the user interface, etc.). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

It will be apparent that systems and/or methods, described herein, may be implemented in different forms of hardware, firmware, and/or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware can be designed to implement the systems and/or methods based on the description herein.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the phrase “only one” or similar language is used. Also, as used herein, the terms “has,” “have,” “having,” and the like are intended to be open-ended terms. Further, the phrase “based on” is intended to mean “based, at least in part, on” unless explicitly stated otherwise.

What is claimed is:

1. A device for noninvasive biological function monitoring, comprising:
a light-guiding component to guide light energy toward tissue to cause the light energy to be absorbed by the tissue;
an ultrasound transmission component to transmit acoustic energy toward the tissue to cause a biological response from the tissue; and
a sensing component to perform one or more of ultrasound or photoacoustic imaging to sense the biological response from the tissue and permit a status of the tissue to be determined,
wherein the biological response is sensed based on the light energy absorbed by the tissue during the biological response caused by the acoustic energy transmitted toward the tissue.

2. The device of claim 1, further comprising:
a light source to emit the light energy that the light-guiding component guides toward the tissue as pulsed light energy at multiple near-infrared range (NIR) wavelengths.

3. The device of claim 2, wherein the light source includes one or more pulsed light-emitting diodes (LEDs) or pulsed laser diodes (PLDs).

4. The device of claim 2, wherein the ultrasound transmission component, the sensing component, and the light source are included in a housing that is wearable on an abdomen or other external body region relative to the tissue associated with the status to be determined.

5. The device of claim 2, further comprising:
a hybrid power supply that comprises one or more batteries, included in or coupled to a housing that includes the ultrasound transmission component, the sensing component, and the light source, to receive and store electrical energy from a wired power source.

6. The device of claim 5, wherein the one or more batteries are configured to deliver the stored electrical energy to one or more of the ultrasound transmission component, the sensing component, or the light source when the wired power source is unavailable.

7. The device of claim 2, wherein the light-guiding component comprises:
an optical fiber bundle to guide the light energy emitted by the light source; and
a housing that comprises a transparent or translucent material that enables the light energy to be emitted through the housing toward the tissue.

8. The device of claim 7, wherein the light-guiding component further comprises:
a rubberized ring associated with a structural pattern to indicate a position and an angle of the light-guiding component in one or more of the ultrasound or photoacoustic imaging used to sense the biological response from the tissue.

9. A system for biological function monitoring, comprising:
a photoacoustic monitoring device comprising:
a light source;
a light-guiding component;
an ultrasound transmission component; and
a sensing component; and
a control device, wherein the control device includes one or more processors to:
control the light source to emit light energy as pulsed light energy at multiple near-infrared range (NIR) wavelengths,
wherein the light-guiding component is arranged to guide the light energy emitted by the light source toward tissue of a patient;
control the ultrasound transmission component to transmit acoustic energy toward the tissue to incite a biological response from the tissue;
receive, from the ultrasound sensing component, one or more of ultrasound or photoacoustic imaging data associated with the biological response,
wherein the imaging data is representative of the light energy being absorbed by the tissue during the biological response; and
generate an output that indicates the biological response from the tissue of the patient.

10. The system of claim 9, wherein the light source includes one or more pulsed light-emitting diodes (LEDs) or pulsed laser diodes (PLDs).

11. The system of claim 9, wherein the ultrasound transmission component, the sensing component, and the light source are included in a housing that is wearable on an abdomen or other external body region relative to the tissue associated with the imaging data.

12. The system of claim 11, further comprising:
a hybrid power supply that comprises one or more batteries, included in or coupled to the housing that includes the ultrasound transmission component, the sensing component, and the light source, to receive and store electrical energy from a wired power source.

13. The system of claim 12, wherein the one or more batteries are configured to deliver the stored electrical energy to one or more of the ultrasound transmission component, the sensing component, or the light source when the wired power source is unavailable.

14. The system of claim 9, wherein the light-guiding component comprises:
an optical fiber bundle to guide the light energy emitted by the light source; and
a housing that comprises a transparent or translucent material that enables the light energy to be emitted through the housing toward the tissue.

15. The system of claim 14, wherein the light-guiding component further comprises:
a rubberized ring associated with a structural pattern to indicate a position and an angle of the light-guiding component in the imaging data.

16. A method for monitoring a biological function, comprising:
causing, by a control device, a light source of a monitoring device to emit pulsed light energy at multiple near-infrared range (NIR) wavelengths,
wherein the light source is coupled to a light-guiding component that guides the light energy toward tissue of a patient to cause the light energy to be absorbed by the tissue;
causing, by the control device, an ultrasound transmission component of the monitoring device to transmit acoustic energy toward the tissue to cause a biological response from the tissue;
obtaining, by the control device and from a sensing component of the monitoring device, one or more of ultrasound or photoacoustic imaging data associated with the biological response,
wherein the ultrasound or photoacoustic imaging data is generated from the light energy being absorbed by the tissue; and
generating, by the control device, an output that indicates the biological response indicated in the ultrasound or photoacoustic imaging data.

17. The method of claim 16, wherein the light source includes one or more pulsed light-emitting diodes (LEDs) or pulsed laser diodes (PLDs).

18. The method of claim 16, wherein the ultrasound transmission component, the sensing component, and the light source are included in a housing that is wearable on an abdomen or other external body region relative to the tissue associated with the ultrasound or photoacoustic imaging data.

19. The method of claim 16, wherein the monitoring device includes one or more batteries, included in or coupled to a housing that includes the ultrasound transmission component, the sensing component, and the light source, to receive and store electrical energy.

20. The method of claim 16, wherein the ultrasound or photoacoustic imaging data indicates a position and an angle of a rubberized ring of the light-guiding component.
